(19) United States
(12) Patent Application Publication
(10) Pub. No.: US 2011/0300093 A1
(43) Pub. Date: Dec. 8, 2011

(54) COSMETIC COMPOSITION CONTAINING AN AMPHIPHILIC ZWITTERIONIC COPOLYMER

(75) Inventors: Denis Bendejacq, Paris (FR); Chi-Thanh Vuong, Lognes (FR)

(73) Assignee: Rhodia Operations, Aubervilliers (FR)

(21) Appl. No.: 13/123,687

(22) PCT Filed: Oct. 8, 2009
(86) PCT No.: PCT/EP09/63127

§ 371 Date: Aug. 26, 2011

(30) Foreign Application Priority Data
Oct. 10, 2008 (FR) 08/05609

(51) Int. Cl. A61K 8/72; A61Q 19/10; A61Q 5/12; A61K 31/685; A61Q 5/02

(52) U.S. Cl. 424/70.16; 424/70.11; 514/77

(57) ABSTRACT

The present invention relates to a cosmetic composition that includes an amphiphilic zwitterionic copolymer. The amphiphilic zwitterionic copolymer can particularly be used for imparting rheological properties, in particular a beneficial texture, to said composition and/or to the foam resulting from said composition.

The present invention relates to a cosmetic composition comprising a zwitterionic amphiphilic copolymer. The zwitterionic amphiphilic copolymer may especially contribute towards giving the composition and/or the foam derived from the composition rheological properties, especially an interesting texture.

Cosmetic compositions, especially aqueous cosmetic compositions, especially rinse-out compositions such as shampoos, certain hair conditioners and shower gels, generally have certain rheological and/or texture properties suitable for:
flow
suspension and/or physical stability
a sensory profile and/or a feel appreciated by consumers; especially during handling and application outside of the packaging, and/or
a combination and/or compromise of these properties and/or textures.

Many commercial rinse-out cosmetic compositions comprise a surfactant system comprising an ionic, for example anionic, surfactant and optionally a salt and/or a structuring agent, in the form of organized phases of surfactant(s). These organized phases allow the production of compositions of relatively high viscosity, which are judged as suitable for compositions of this type. However, to obtain organized phases and/or to obtain the viscosity judged as suitable, it is necessary to use relatively large amounts of surfactant(s) and/or of salt(s) and/or of structuring agent(s), which may make the composition relatively irritant and/or aggressive to the skin the scalp and/or the hair. These relatively large amounts may moreover be perceived as environmentally and/or toxicologically unfriendly. These relatively large amounts may moreover make the compositions expensive. There is a need for compositions that have reduced amounts of products, while at the same time maintaining rheological properties and/or a suitable texture. There is also a need for compositions of more limited cost. There is also a need for compositions of modified texture and/or modified rheology.

Document WO 03/054350 (Rhodia) describes shampoos comprising at least 5% by weight of a surfactant system in the form of giant micelles in the presence of salt and a charged amphiphilic copolymer, preferably comprising at least 5 mol % of hydrophobic units. Said document teaches that sufficient contents are capable of tripling the viscosity. The examples show compositions comprising 1% of a terpolymer comprising hydrophobic units (24 mol % of ethylhexyl acrylate EHA), anionic units (53 mol % of acrylic acid AA) and cationic units (23 mol % of MES), 1% of NaCl and a surfactant system. There is, however, a need for different and/or more efficient solutions for modifying the rheology and/or the texture, especially with lower contents of copolymer and/or with lower contents of hydrophobic units, and/or with lower contents of surfactant(s) and/or of salt(s),

Document U.S. Pat. No. 4,994,088 (Kao) describes amphoteric copolymers comprising cationic units and anionic units, and describes zwitterionic homopolymers containing a betaine group. The document indicates that these polymers may afford or contribute toward affording hair-conditioning properties. However, the zwitterionic homopolymers are not tested in compositions comprising surfactants. There remains a need to improve and/or modify the rheological and/or texture properties.

Document EP 1 064 915 (L'Oréal) describes amphoteric terpolymers comprising cationic units, anionic units and hydrophobic units. The document indicates that these polymers may afford hair-conditioning properties. There remains a need to improve and/or modify the rheological and/or texture properties.

Documents U.S. Pat. No. 5,139,037 and U.S. Pat. No. 5,089,252 (L′Oréal) describe compositions comprising amphiphilic copolymers comprising hydrophobic units, and zwitterionic units of carboxybetaine type, such as the Amphoset copolymers from Mitsibishi or the Amersette copolymers from Amercol. Units of carboxybetaine type are, however, very pH-sensitive and do not allow easy use of the copolymer. Such units make the copolymer difficult to formulate and sparingly modulable. The documents indicate that these polymers can afford or contribute toward affording hair-conditioning properties. There remains a need to improve and/or modify the rheological and/or texture properties, with ingredients that are easy to formulate.

Document WO 97/12596 (L'Oréal) describes compositions comprising amphiphilic copolymers comprising hydrophobic units, and zwitterionic units of carboxybetaine type, such as the Yukaformer AM74 copolymer from Mitsibishi. Units of carboxybetaine type are, however, very pH-sensitive and do not allow easy use of the copolymer. Such units make the copolymer difficult to formulate and sparingly modulable. The document indicates that these polymers can afford or contribute toward affording hair-conditioning properties. There remains a need to improve and/or modify the rheological and/or texture properties, with ingredients that are easy to formulate.

Document WO 97/12596 (L'Oréal) describes compositions comprising copolymers comprising zwitterionic units. The copolymers used are not amphiphilic, they do not comprise hydrophobic units. The document indicates that these polymers can afford or contribute toward affording hair-conditioning properties. There remains a need to improve and/or modify the rheological and/or texture properties.

The present invention satisfies at least one of the needs mentioned above, by proposing an aqueous cosmetic composition comprising:
a) a surfactant system,
b) optionally at least one salt,
c) at least one copolymer comprising:
   units A_{Z} comprising a betaine group, the betaine group comprising a cationic group and an anionic group other than a carboxylate group,
   hydrophobic units B (B_{phobic}) and/or amphiphilic units (Bₐₘₚₕᵢ), and
   optionally other hydrophilic units Aₒₜₕₑᵣ.

The invention also relates to the use of the copolymer c) in an aqueous cosmetic composition comprising a) a surfactant system, and b) optionally at least one salt. The copolymer c) may be used as suspension agent, or as agent for modifying the rheology and/or the texture of the composition and/or the foam generated from the composition, especially as agent for increasing the viscosity or the suspending power or the suspension stability. The invention also relates to a process for modifying the rheology of an aqueous cosmetic composition, in which the copolymer c) is mixed with a) a surfactant system, b) optionally at least one salt and optionally other ingredients. The invention also relates to a process for preparing an aqueous cosmetic composition, in which a) a surfactant system, b) optionally at least one salt and the copolymer c) and optionally other ingredients are mixed together, optionally with intermediate premixing steps.

The compositions of the invention may especially have very particular rheological and/or texture properties, or effects on touching or handling. They may especially have the qualities of a viscoelastic gel (G′>G″ in which G′ is the storage modulus and G″ is the loss modulus, over a frequency range between 1 and 10 Hz, with rheometry geometry of cone-plate type; the modulus values being measured in the linear viscoelastic regime, at 25° C., for example with a Carrimed rheometer). They may especially have a transition from the viscoelastic gel state (G>G″) to the liquid state (G′<G″) at temperatures close to body temperature, for example between 35° C. and 39° C., typically at about 37° C. This property may give the user a pleasant sensation and/or may be perceived as positive during touching and/or handling, especially during application to the skin and/or the hair. The compositions may especially have viscoelastic gel rheology at normal force, i.e. reacting to shear by a force perpendicular to the shear plane. This property may give the user a pleasant sensation and/or may be perceived as positive on touching and/or handling, especially on application to the skin and/or the hair, the user perceiving it as a persistance or a resistance during application. The invention also relates to cosmetic compositions that are viscoelastic at normal force, whether or not they comprise the copolymer c), the surfactant system a) and the optional salt b) or a combination thereof. In the case where foam is generated from the composition, this foam may have an elastic nature, which is pleasant to the user.

### DEFINITIONS

In the present patent application, organized phases of surfactants denotes organized structures of surfactants other than spherical micelles. They may especially be more or less long cylindrical micelles (for example with a length at least four times and preferably at least 10 times the diameter). They may, for example, be giant micelles (worm-like micelles). They may be lamellar phases. They may be phases of monolamellar or multilamellar vesicle type (also referred to in the literature as spherulites), phases having both a lamellar phase and spherulites, and hexagonal or cubic phases. They may be SSLs or Structured Surfactant Liquids. Organized phases of surfactants are generally characterized by a large increase in viscosity relative to a liquid-order spherical micelle phase (with no long-range order). The transition from a spherical micelle phase to an organized phase may be generated by an increase in surfactant content, by adding a structuring agent or by increasing its content (it may be a surfactant) or by adding a salt or a combination of these types of measure. It is mentioned that the presence of organized phases does not exclude the presence of spherical micelles. Such organized phases are known to those skilled in the art and many suitable compositions are described in the literature. Suitable characterization modes are also described. Viscosity measurements, phase diagrams, polarized-light optical microscopy, or observations of cryofractures may especially be used. In the present patent application, the term salt preferably means an inorganic salt or a salt not comprising more than 6, preferably not more than 5, preferably not more than 4, preferably not more than 3, preferably not more than 2 and preferably not more than 1 carbon atom(s). In the present patent application, the term “structuring agent” means an organic molecule that makes it possible to generate organized phases. It may be a surfactant of low HLB, for example less than 20, preferably less than 15 and especially less than 10, which is preferably nonionic. In the present patent application, structuring agents are considered as forming part of the surfactant system. Structuring agents are known to those skilled in the art.

In the present patent application, the term monomer-based unit for the various units of the units A_{Z precursor}, denotes a unit that may be obtained directly from said monomer by polymerization. Thus, for example, a unit derived from an acrylic or methacrylic acid ester does not cover a unit of formula —CH₂—CH(COOH)—, —CH₂—C(CH₃)(COOH)— or —CH₂—CH(OH)—, respectively, obtained, for example, by polymerizing an acrylic or methacrylic acid ester, or vinyl acetate, respectively, and then by hydrolyzing. A unit derived from acrylic or methacrylic acid covers, for example, a unit obtained by polymerizing a monomer (for example an acrylic or methacrylic acid ester) and then by reacting (for example by hydrolysis) the polymer obtained so as to obtain units of formula —CH₂—CH(COOH)— or —CH₂—C(CH₃)(COOH)—. A unit derived from a vinyl alcohol covers, for example, a unit obtained by polymerizing a monomer (for example a vinyl ester) and then by reacting (for example by hydrolysis) the polymer obtained so as to obtain units of formula —CH₂—CH(OH)—. Units derived from a monomer A_{Z} may, for example, have been obtained by polymerization of monomers A_{Z precursors} followed by post-polymerization reaction to obtain units comprising the betaine group. The units A_{Z} are not considered as monomer-based units A_{Z precursor} not comprising a betaine group.

In the present patent application, the term “hydrophobic” is used in its usual sense as “which has no affinity for water”; this means that the organic polymer from which it is formed, taken alone (of the same composition and of the same molar mass), would form a macroscopic two-phase solution in distilled water at 25° C., at a concentration of greater than 1% by weight.

In the present patent application, the terms “hydrophilic” “water-soluble” and “water-dispersible” are also used in their usual sense as “which has affinity for water”, i.e. not capable of forming a macroscopic two-phase solution in distilled water at 25° C. at a concentration of greater than 1% by weight.

The term cationic or potentially cationic units A_{C} means units that comprise a cationic or potentially cationic group. The cationic units or groups are units or groups that contain at least one positive charge (generally combined with one or more anions such as the chloride ion, the bromide ion, a sulfate group or a methyl sulfate group), irrespective of the pH of the medium into which the copolymer is introduced. Potentially cationic units or groups are units or groups that may be neutral or may bear at least one positive charge depending on the pH of the medium into which the copolymer is introduced. In this case, they will be referred to as potentially cationic units in neutral form or in cationic form. By extension, it is possible to speak of cationic or potentially cationic monomers.

The term anionic or potentially anionic units A_{A} means units that comprise an anionic or potentially anionic group. Anionic units or groups are units or groups that bear at least one negative charge (generally combined with one or more cations such as cations of alkali metal or alkaline-earth metal compounds, for example sodium, or with one or more cationic compounds such as ammonium), irrespective of the pH of the medium in which the copolymer is present. Potentially anionic units or groups are units or groups that may be neutral or may bear at least one negative charge depending on the pH of the medium in which the copolymer is present. In this case, they will be referred to as potentially anionic units A_{A} in neutral form or in anionic form. By extension, it is possible to speak of anionic or potentially anionic monomers.

The term neutral units A_{N} means units that do not bear a charge, irrespective of the pH of the medium in which the copolymer is present.

In the present patent application, unless otherwise indicated, when the molar mass is referred to, it is the absolute mass-average molar mass, expressed in g/mol. It may be determined by aqueous gel permeation chromatography (GPC), by light scattering (DDL or alternatively MALLS for an aqueous eluent), with an aqueous eluent or an organic eluent (for example formamide) depending on the composition of the polymer.

In the present patent application, unless otherwise mentioned, the amounts and proportions are indicated as active material (as opposed to the dilute or dispersed material), and on a weight basis.

During a micellar polymerization, micelles comprising hydrophobic monomers (B_{phobic}) and/or amphiphilic monomers (B_{phobic}) are formed in an aqueous fluid. The number of monomers in these micelles is noted as n_{H}. The micelles may be micelles of a nonpolymerizable surfactant compound, with a hydrophobic monomer (B_{phobic}) and/or amphiphilic monomer (B_{phobic}) included in the micelles. The micelles may be formed from an amphiphilic monomer forming micelles by self-association at the amount in which it is used, said micelles also not comprising any hydrophobic monomer. The micelles may be formed from an amphiphilic monomer (Bₐₘₚₕᵢ) forming micelles by self-association at the amount at which it is used, said micelles also comprising a hydrophobic monomer inside. The micelles may comprise a nonpolymerizable surfactant and an amphiphilic monomer (Bₐₘₚₕᵢ) whose association makes it possible to form micelles (co-micellization), said micelles also not comprising any hydrophobic monomers. The micelles may comprise a nonpolymerizable surfactant and an amphiphilic monomer (Bₐₘₚₕᵢ) whose association makes it possible to form micelles (co-micellization), said micelles also comprising a hydrophobic monomer (B_{phobic}) inside. The number n_{H} corresponds to the total number of hydrophobic monomers (B_{phobic}) in the micelle, when the micelle comprises only hydrophobic monomers (B_{phobic}).

The number n_{N} may be evaluated as taught in document P. Kujawa: J. M. Rosiak; J. Seib; F. Candau Macromolecular Chem. & Physics, 202, 8, 1384-1397, 2001:

$${n}_{H} = \frac{{Monomer }^{r} e ] \times {N}_{agg}}{surfactant if ] - CMC}$$

in which:
[Monomer] is the (molar) concentration of hydrophobic and/or amphiphilic monomer
N_{agg} is the aggregation number of the surfactant
[surfactant] is the (molar) concentration of surfactant or the sum of the molar concentrations of surfactant and of amphiphilic monomer if the latter participates in the formation of the micelle,
CMC is the critical micelle concentration (molar) of the surfactant or of the amphiphilic monomer or of the combination of the amphiphilic monomer and of the surfactant, if the surfactant monomer participates in the formation of the micelle.

The critical micelle concentrations and the aggregation numbers are the ones most commonly known in the literature. Alternatively, they may be evaluated via the protocol described in P. Becher J. Colloid Sci. 16, 49, 1961.

As values that are useful for determining certain n_{H} values, mention is made especially of:

Molar masses;
Sodium dodecyl sulfate (“SDS”): Mw SDS=288 g/mol
Lauryl methacrylate=254 g/mol

CMC and aggregation number for sodium dodecyl sulfate
cmc SDS=0.007 mol/L=02 weight %
N_{agg} SDS=62
Reference; “Remove detergent from protein samples” Technical Resource TR0019.0
Pierce, 3747 N. Meridian Road
P.O. Box 117
Rockford, Ill. 61105

It may be considered that the number of hydrophobic and/or amphiphilic units in a group B is equal on average to the number of monomers included in a micelle, said micelles being formed by performing a controlled micelle polymerization. However, it is not excluded to perform other types of polymerization, for example block radical polymerizations, preferably with at least 3 blocks, preferably at least 5, in a controlled manner, taking into account the number n_{H} of monomers engaged per macromolecular chain during the relevant blocks.

### Copolymer c)

The copolymer c) comprises:
units A_{Z} comprising a betaine group, the betaine group comprising a cationic group and an anionic group other than a carboxylate group,
hydrophobic units (B_{phobic}) and/or amphiphilic units (Bₐₘₚₕᵢ), and
optionally other hydrophilic units Aₒₜₕₑᵣ.

The copolymer c) is preferably a copolymer obtained via a controlled micelle polymerization process.

According to one particular mode of the invention, the copolymer c) comprises:
a macromolecular chain A comprising units AZ comprising a betaine group, the betaine group comprising a cationic group and an anionic group other than a carboxylate group, and optionally other hydrophilic units Aₒₜₕₑᵣ.
at least one group B of hydrophobic units (B_{phobic}) and/or amphiphilic units (Bₐₘₚₕᵢ).

It is noted that the number n_{H} of hydrophobic units in the group B may especially be
less than 2.6 or
greater than or equal to 2.6.

The number n_{H} is preferably between 2 and 20, preferably between 4 and 15 and preferably between 5 and 12. The copolymer c) proves to be more efficient in terms of rheological effect in these ranges. The number n_{H} may especially be other than 2.6, 3, 5 or 10.

The macromolecular chain A is typically a linear macromolecular chain of units comprising the betaine group and other optional hydrophilic groups. This macromolecular chain is interrupted with (or sectioned by) groups B of hydrophobic and/or amphiphilic units. The groups B with the macromolecular chain A typically form a linear macromolecular chain, known as a “complete chain”. Such a macromolecular chain (“complete chain”) may typically be obtained by controlled micelle polymerization. The number of units in the groups B is noted n_{H}. This number may be varied as a function of the process chosen and of the operating conditions chosen (especially by the amounts and ratios of monomers used and/or by the natures, amounts and ratios of surfactants used in the polymerization process). The number of groups B may also be varied as a function of the chosen process and of the chosen operating conditions (especially by the amounts and ratios of monomers and/or initiators used and/or by the natures, amounts and ratios of surfactants used during the polymerization process).

The units A_{Z} comprising a betaine group and optionally the other hydrophilic units Aₒₜₕₑᵣ preferably form a macromolecular chain A containing a hydrocarbon-based polyalkylene chain optionally interrupted with one or more nitrogen or sulfur atoms (it being possible for such atoms not to interrupt the macromolecular chain). The complete chain preferably forms a macromolecular chain containing a hydrocarbon-based polyalkylene chain optionally interrupted with one or more nitrogen or sulfur atoms (it being possible for such atoms not to interrupt the macromolecular chain). The optionally interrupted hydrocarbon-based polyalkylene chain is likened to a backbone of macromolecular chains, said macromolecular chains generally comprising side groups of the other units, in particular betaine groups.

The groups B, generally of a number greater than or equal to 2 (there are several), may typically interrupt the macromolecular chain A in a statistical manner. Thus, along the complete chain, the groups B, separated by the units constituting the macromolecular chain A, may be more or less spaced apart, the space distribution typically being statistical. It is noted that groups B may be present at the end of the complete chain. However, this is not particularly desired, and usually the groups B will not be present at the end of the complete chain (more than 50% by weight of the complete chains do not comprise groups B at the end of the chain).

The number of groups B may especially be greater than or equal to 2, preferably greater than or equal to 3, for example greater than or equal to 5 or even 10. The complete chain may thus especially resemble a multiblock chain with a number of blocks of greater than or equal to 4, preferably greater than or equal to 5, preferably greater than or equal to 6, for example greater than or equal to 9 or 10 or 11, or even greater than or equal to 19 or 20 or 21.

The macromolecular chain A is typically water-soluble, i.e. a polymer formed solely from units of the macromolecular chain A, without the groups B, of similar average molecular mass (for example obtained under the same polymerization conditions, especially with the same initiator/monomer ratio with the same operating conditions), would be water-soluble (at 25° C. and at 1% by weight),

The number n_{N} is preferably less than 100, preferably less than 50 and preferably less than 25. It may, for example, be between 3 and 50 and preferably between 5 and 30, for example between 10 and 25.

### Units A_{Z}

The betaine group of the units A_{Z} comprises an anionic group and a cationic group. The anionic group is other than a carboxylate group (—COO⁻ or —COOH in acid form). The betaine group is thus other than a carboxybetaine group. It may especially be a sulfur group such as a sultanate group, a phosphorus group such as a phosphate, phosphonate or phospinate group, or an ethenolate group. It is preferably a sulfonate group. The cationic group may be an onium or inium group of the nitrogen, phosporus or sulfur family, for example an ammonium, pyridinium, phosphonium or sulfonium group. Preferably, it is an ammonium group (which is preferably quaternary). The betaine group may especially be a sulfobetaine or phosphonobetaine group. Advantageously, the betaine group is a sulfobetaine group comprising a sulfonate group and a quaternary ammonium group. It is noted that it would not constitute a departure from the context of the invention to combine several different betaine groups, by combining in the copolymer several different units A_{Z}.

The betaine groups are typically side groups on the copolymer, typically obtained from monomers comprising at least one ethylenic unsaturation

Within the units A_{Z}, the number of positive charges is equal to the number of negative charges. The units A_{Z} are electrically neutral, within at least one pH range.

Useful betaine groups may be represented, in the case of cations of the nitrogen family, by formulae (I) to (V) bearing a cationic charge at the center of the function and an anionic charge at the end of the function and of formula (VI) bearing an anionic charge at the center of the function and a cationic charge at the end of the function, as follows:

—N⁽⁺⁾(R¹)(R²)—R-A-O⁽⁻⁾ (I)

—(R³)C═N⁽⁺⁾(R⁴)—R-A-O⁽⁻⁾ (II)

—(R³)(R)C—N⁽⁺⁾(R⁴)(R⁵)—R-A-O⁽⁻⁾ (III)

—N⁽⁺⁾(═R⁶)—R-A-O⁽⁻⁾ (IV)

—R-A′(—O⁽⁻⁾)—R—N⁽⁺⁾(R¹)(R²)(R⁷) (V)
in which formulae (I) to (IV):
   the symbols R¹, R² and R⁵, which may be identical or different, represent an alkyl radical containing from 1 to 7 carbon atoms and preferably from 1 to 2 carbon atoms,
   the symbols R³ and R⁴ represent hydrocarbon-based radicals forming with the nitrogen atom a nitrogen heterocycle optionally comprising one or more other heteroatoms, especially nitrogen,
   the symbol R⁶ represents a hydrocarbon-based radical forming with the nitrogen atom a saturated or unsaturated nitrogen heterocycle, optionally comprising one or more heteroatoms, especially nitrogen,
   the symbol R represents a linear or branched alkylene radical comprising from 1 to 15 carbon atoms and preferably from 2 to 4 carbon atoms, optionally substituted with one or more hydroxyl groups, or a benzylene radical,
   the symbol A represents S(═O)(═O) OP(═O)(═O), OP(═O)(OR′), P(═O)(OR′) or P(═O)(R′), in which R′ represents an alkyl radical containing from 1 to 7 carbon atoms or a phenyl radical
in which formula (V),
   the symbols R¹ and R² have the definition given above
   the symbols R⁷, which may be identical to or different than R¹ or R², represent an alkyl radical containing from 1 to 7 carbon atoms and preferably from 1 to 2 carbon atoms
   the symbol A′ represents —O—P(═O)—O—,

In the case of cations of the phosphorus family, mention may be made of the betaine groups of formulae (VI) and (VII);

—P⁽⁺⁾(R¹)(R²)—R-A-O⁻⁾ (VI)

—R-A′(—O⁽⁻⁾)—R—P⁽⁺⁾(R¹)(R²)(R⁷) (VII)
in which formula (VI) the symbols R¹, R², R and A have the definition given above
in which formula (VII)
   the symbols R¹, R², R⁷ and R have the definition given above
   the symbol A′ represents —O—P(═O)—O—

In the case of cations of the sulfur family, mention may be made of the betaine groups of formulae (VIII) and (IX);

—S⁽⁺⁾(R¹)—R-A-O⁽⁻⁾ (VIII)

—R-A′(—O⁽⁻⁾)—R—S⁽⁺⁾(R¹)(R²) (IX)
in which formula (VIII) the symbols R and A have the definition given above
in which formula (IX)
   the symbols R¹, R² and R have the definition given above
   the symbol A′ represents —O—P(═O)—O—.

The betaine groups may be linked to the carbon atoms of a macromolecular chain A of the copolymer especially via a divalent or polyvalent hydrocarbon-based unit (for example alkylene or arylene) optionally interrupted with one or more heteroatoms, especially oxygen or nitrogen, an ester unit, an amide unit, or a valency bond.

The copolymer may especially be obtained by radical polymerization
of monomers A_{Z} comprising an ethylenically unsaturated betaine group, especially of ethylenically unsaturated monomers bearing at least one betaine group of formulae (I) to (IX) above,
and of other monomers, comprising monomers B (monomers B_{phobic} and/or Bₐₘₚₕᵢ) from which are derived the hydrophobic units B (B_{phobic}) and/or amphiphilic units B (Bₐₘₚₕᵢ) of the groups B, and optionally other monomers Aₒₜₕₑᵣ from which are derived the optional hydrophilic units Aₒₜₕₑᵣ of the macromolecular chains A.

Said monomers A_{Z} may bear, for example:
one or more monoethylenically or polyethylenically unsaturated hydrocarbon-based radicals (especially vinyl, allyl, styrenyl, etc.),
one or more monoethylenically or polyethylenically unsaturated ester radicals (especially acrylate, methacrylate, maleate, etc.), and/or
one or more monoethylenically or polyethylenically unsaturated amide radicals (especially acrylamido, methacrylamido, etc.).

The units A_{Z} may be derived from at least one betaine monomer A_{Z} selected from the group formed from the following monomers:
alkyl sulfonates or phosphonates of dialkylammonium alkyl acrylates or methacrylates, acrylamido or methacrylamido, such as:
   sulfopropyl dimethyl ammonium ethyl methacrylate, sold by Raschig under the name SPE:

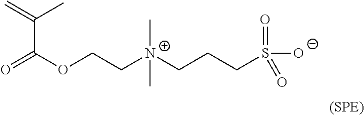

sulfoethyl dimethyl ammonium ethyl methacrylate and sulfobutyl dimethyl ammonium ethyl methacrylate:

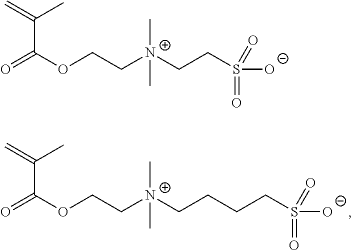

the synthesis of which is described in the article “Sulfobetaine Zwitterionomers based on n-butyl acrylate and 2-Ethoxyethyl acrylate: monomer synthesis and copolymerization behavior, Journal of Polymer Science 40, 511-523 (2002).
sulfohydroxypropyl dimethyl ammonium ethyl methacrylate:

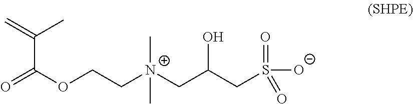

sulfopropyl dimethylammonium propyl acrylamide:

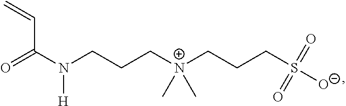

the synthesis of which is described in the article “Synthesis and solubility of the poly(sulfobetaine)s and the corresponding cationic polymer: 1. Synthesis and characterization of sulfobetaines and the corresponding cationic monomers by nuclear magnetic resonance spectra”, Wen-Fu Lee and Chan-Chang Tsai, Polymer, 35 (10), 2210-2217 (1994),
sulfopropyl diethylammonium propyl methacrylamide, sold by Raschig under he name SPP:

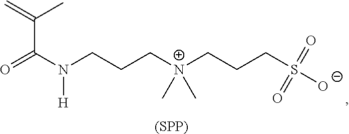

sulfopropyl diethylammonium ethyl methacrylate, sold by Raschig under the name SPDA:

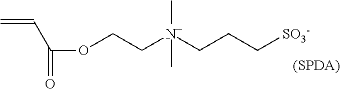

sulfohydroxypropyl dimethyl ammonium propyl methacrylamido

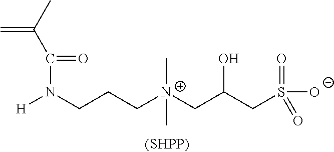

sulfopropyl diethyl ammonium ethyl methacrylate:

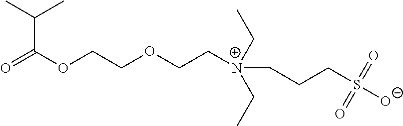

the synthesis of which is described in the article “Poly(sulphopropylbetaines): 1. Synthesis and characterization”, V. M. Monroy Soto and J. C. Galin, Polymer, 1984, Vol 25, 121-128.
sulfohydroxypropyl diethyl ammonium ethyl methacrylate:

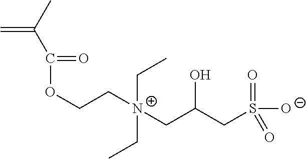

heterocyclic betaine monomers, such
   piperazine-based sulfobetaines:

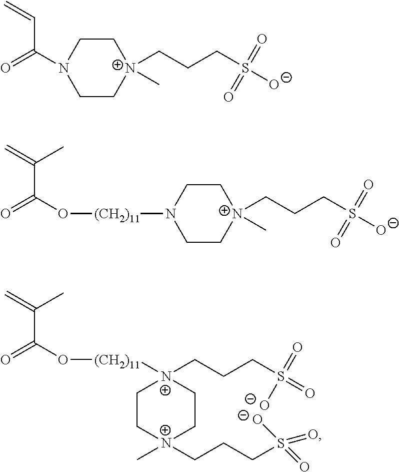

the synthesis of which is described in the article “Hydrophobically Modified Zwitterionic Polymers: Synthesis, Bulk Properties, and Miscibility with Inorganic Salts”, P, Koberle and A. Laschewsky, Macromolecules 27, 2165-2173 (1994)
sulfobetaines derived from 2-vinylpyridine and 4-vinylpyridine, such as
   2-vinyl(3-sulfopropyl)pyridinium betaine (2SPV or “SPV”), sold by Raschig under the name SPV,

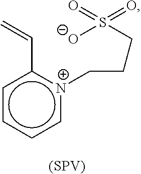

4-vinyl(3-sulfopropyl)pyridinium betaine (4SPV), the synthesis of which is described in the article “Evidence of ionic aggregates in some ampholytic polymers by transmission electron microscopy”, V. M. Castaño and A. E. González, J. Cardoso, O. Manero and V. M. Monroy, J. Mater. Res., 5 (3), 654-657 (1990):

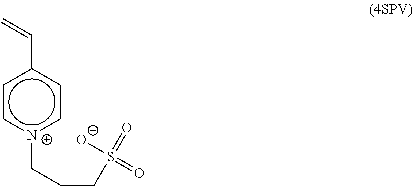

1-vinyl-3-(3-sulfopropyl)imidazolium betaine:

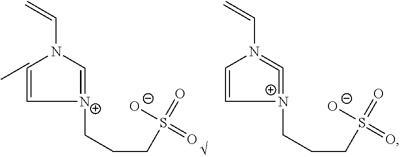

the synthesis of which is described in the article “Aqueous solution properties of a polyvinyl imidazolium sulphobetaine)”, J. C. Salamone, W. Volkson, A. P. Oison, S. C. Israel, Polymer, 19, 1157-1162 (1978)
dialkylammonium alkyl allyl alkylsulfonates or phosphonates, for instance sulfopropyl methyl diallyl ammonium betaine:

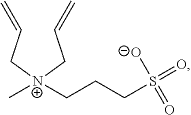

the synthesis of which is described in the article “New poly(carbobetaine)s made from zwitterionic diallylammonium monomers”, Favresse, Philippe; Laschewsky, Andre, Macromolecular Chemistry and Physics, 200(4), 887-895 (1999).
dialkylammonium alkyl styrene alkylsulfonates or phosphonates, for instance:

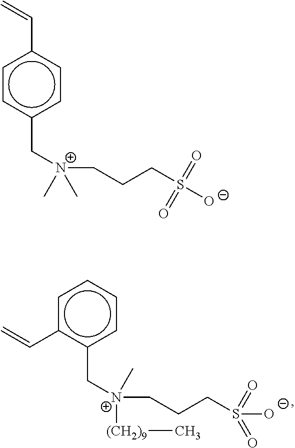

the synthesis of which is described in the article “Hydrophobically Modified Zwitterionic Polymers: Synthesis, Bulk Properties, and Miscibility with Inorganic Salts”, P. Koberle and A. Laschewsky, Macromolecules 27, 2165-2173 (1994).
betaines derived from ethylenically unsaturated dienes and anhydrides, such as:

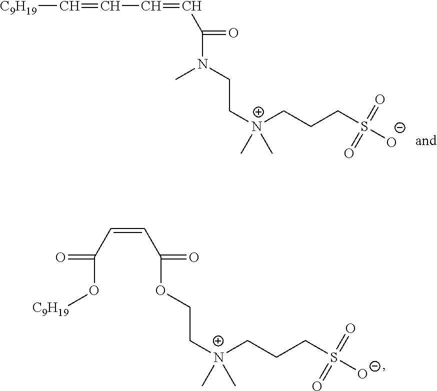

the synthesis of which is described in the article “Hydrophobically Modified Zwitterionic Polymers: Synthesis, Bulk Properties, and Miscibility with Inorganic Salts”, P. Koberle and A. Laschewsky, Macromolecules 27 2165-2173 (1994)
phosphobetaines, such as:

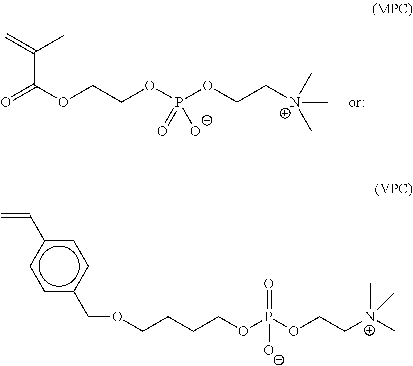

The synthesis of MPC and VPC is described in EP 810 289 B (Biocompatibles, Alister et al.),
betaines derived from cyclic acetals, preferably ((dicyanoethanolate)ethoxy)dimethyl-ammoniumpropylmethacrylamide.

The polymer according to the invention may also be obtained in a known manner by chemical modification of a polymer known as a precursor polymer, comprising units A_{Z precursor} that will be modified (betainized) by post-polymerization reaction to give units A_{Z} containing a betaine group. Thus, sulfobetaine units may be obtained by chemical modification of units of a precursor polymer, preferably by chemical modification of a polymer comprising pendent amine functions, using a sulfonate electrophilic compound, preferably a sultone (propanesultone, butanesultone), or a haloalkylsulfonate.

A few examples of synthesis are given below:

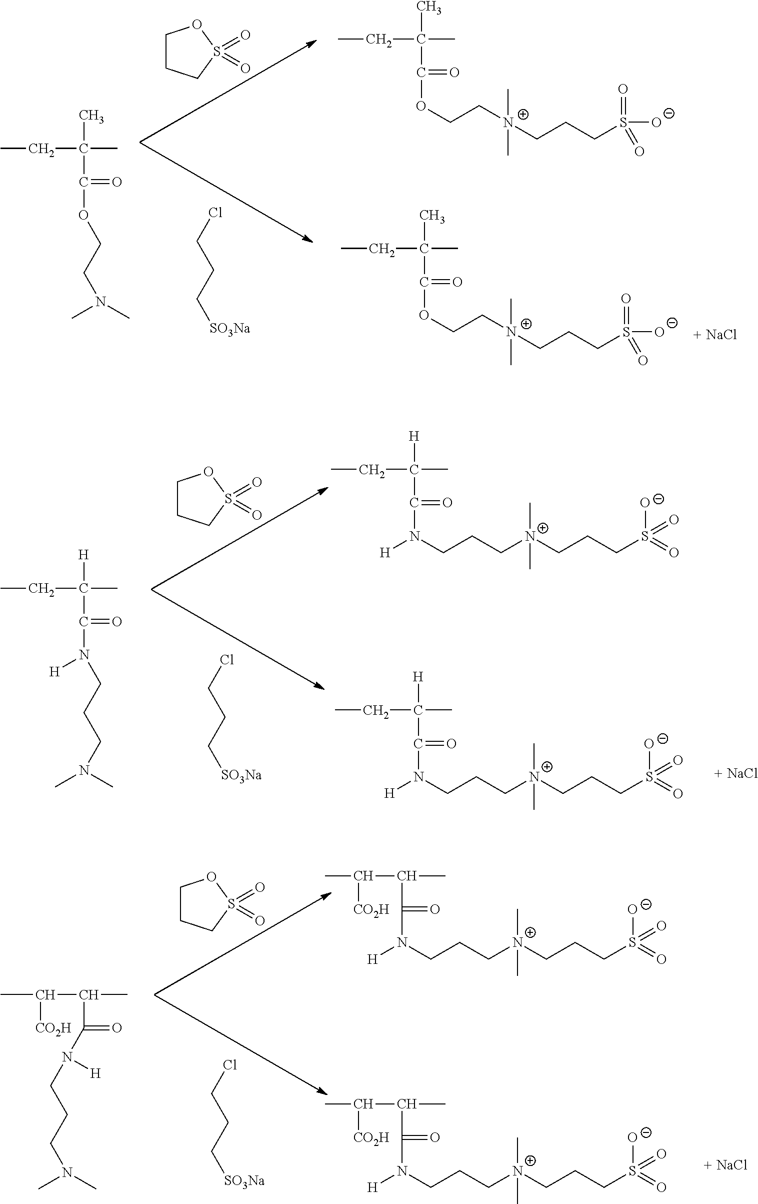

The main routes of access by chemical modification of precursor polymer via sultones and haloalkylsulfonates are described especially in the following documents:
“Synthesis and aqueous solution behaviour of copolymers containing sulfobetaine moieties in side chains”, I. V. Berlinova, I. V. Dimitrov, R. G, Kalinova, N. G. Vladimirov, Polymer 41, 831-837 (2000)
“Poly(sulfobetaine)s and corresponding cationic polymers: 3 Synthesis and dilute aqueous solution properties of poly(sulfobetaine)s derived from styrene-maleic anhydride)”, Wen-Fu Lee and Chun-Hsiung Lee, Polymer 38 (4), 971-979 (1997)
“Poly(sulfobetaine)s and corresponding cationic polymers, VIII, Synthesis and aqueous solution properties of a cationic poly(methyl iodide quaternized styrene-N,N-dimethylaminopropyl maleamidic acid) copolymer”, Lee, Wen-Fu: Chen, Yan-Ming, Journal of Applied Polymer Science 80, 1619-1626 (2001)
“Synthesis of polybetaines with narrow molecular mass distribution and controlled architecture”, Andrew B. Lowe, Norman C. Billingham and Steven P. Armes, Chem. Commun., 1555-1556 (1996)
“Synthesis and Properties of Low-Polydispersity Poly(sulfopropylbetaine)s and Their Block Copolymers”, Andrew B. Lowe, Norman C. Billingham, and Steven P. Armes, Macromolecules 32, 2141-2146 (1999)
Japanese patent application published on 21 Dec. 1999, under number 11-349826.

The preparation of polyphosphonato- and phosphinatobetaines by chemical modification is reported in “New polymeric phosphonato-, phosphinato- and carboxybetaines”, T. Hamaide, Makromolecular Chemistry 187, 1097-1107 (1986).

According to one preferred embodiment, the units A_{Z} have one of the following formulae:

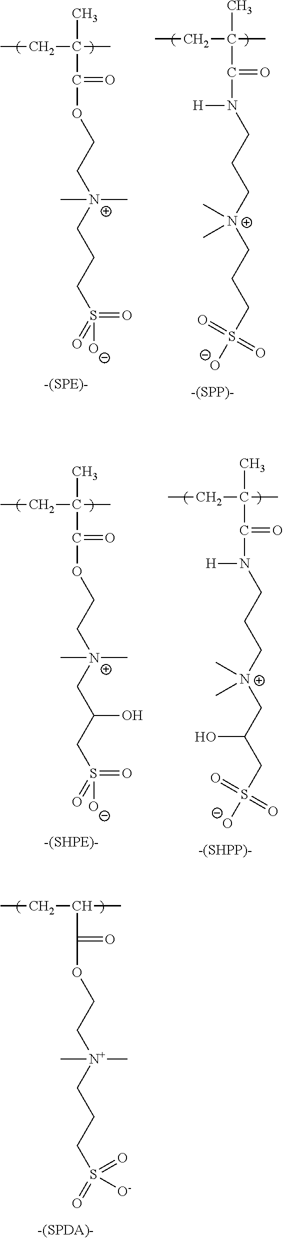

### Units Aₒₜₕₑᵣ

The macromolecular chain A may also comprise hydrophilic units Aₒₜₕₑᵣ. These units are other than the units A_{Z}; they do not comprise betaine groups. The units Aₒₜₕₑᵣ are derived from monomers Aₒₜₕₑᵣ. The units Aₒₜₕₑᵣ may especially comprise:
neutral units A_{N}, derived from neutral monomers A_{N},
cationic or potentially cationic units A_{C}, derived from cationic or potentially cationic monomers A_{C},
anionic or potentially anionic units A_{A}, derived from anionic or potentially anionic monomers A_{A},
mixtures or combinations of such units.

According to particular embodiments, the copolymer is substantially free (it comprises less than 1 mol % and preferably less than 0.5 mol %, and preferably none at all) of the following units:
units A_{C}, and/or
units A_{N} chosen from
   the alkoxy units of the following formula:

—CH₂—CHR⁶[—X²—(CH₂—CH₂—O)ₙ—R⁷]—

in which:
R⁶ is a hydrogen atom or a methyl group,
X² is a group of formula —CO—O—, —CO—NH— or —C₆H₄—CH₂—,
n is an integer or average number greater than or equal to 1,
R⁷ is a hydrogen atom, an alkyl group or a tristyrylphenyl group, and/or
the hydroxyl units of the following formula:

—CH₂—CHR⁸[—X²—R⁸]—
in which:
R⁶ is a hydrogen atom or a methyl group,
X² is a group of formula —CO—O—, —CO—NH— or —C₆C₄—CH₂—,
R⁸ is a hydrocarbon-based group containing at least two carbon atoms, comprising at least two —OH groups, preferably on two consecutive carbon atoms, and/or
hydroxyalkyl acrylate or methacrylate units.

If the copolymer comprises hydrophilic units Aₒₜₕₑᵣ, they will preferably be neutral units A_{N}, without anionic or potentially anionic units A_{A} and/or without cationic or potentially cationic units A_{C}.

### Units A_{N}

The units A_{N} are hydrophilic neutral units. As examples of hydrophilic neutral monomers A_{N} from which the units A_{N} may be derived, mention may be made of:
hydroxyalkyl esters of α,β-ethylenically unsaturated acids such as hydroxyethyl, hydroxypropyl acrylates and methacrylates, glycerol monomethacrylate, etc.,
α,β-ethylenically unsaturated amides such as acrylamide (AM), methacrylamide, N-methylolacrylamide, dimethylacylamide, dimethylmethacrylamide, etc.,
α,β-ethylenically unsaturated monomers bearing a water-soluble polyoxyalkylene segment of the polyethylene oxide type, for instance random or block polyethylene and/or polypropylene oxide α-methacrylates (Bisomer S20W, S10W, etc. from Laporte) or α,ω-dimethacrylates, etc.,
α,β-ethylenically unsaturated monomers that are precursors of hydrophilic units or segments such as vinyl acetate, which, once polymerized, may be hydrolyzed to give rise to vinyl alcohol units or polyvinyl alcohol segments,
vinyllactams, for instance vinylpyrrolidone,
α,β-ethylenically unsaturated monomers of ureido type and in particular 2-imidazolidinone ethyl methacrylamide optionally as a mixture (Sipomer WAM II from Rhodia),
mixtures or combinations thereof.

### Units A_{C}

The units A_{C} are cationic or potentially cationic units comprising 1, 2, 3 or more cationic or potentially cationic groups in the chain forming the copolymer backbone or preferably in a side position relative to the chain forming the copolymer backbone.

The cationic units A_{C} are preferably units comprising at least one quaternary ammonium group. The potentially cationic units A_{C} may be units comprising at least one tertiary amine group.

As examples of potentially cationic monomers A_{C} from which the units A_{C} may be derived, mention may be made of:
α,β-monoethylenically unsaturated carboxylic acid N,N-(dialkylamino-ω-alkyl)amides, such as N,N-dimethylaminomethyl-acrylamide or -methacrylamide, 2-(N,N-dimethylamino)ethyl-acrylamide or -methacrylamide, 3-(N,N-dimethylamino)propyl-acrylamide or -methacrylamide, 4-(N,N-dimethylamino)butyl-acrylamide or -methacrylamide,
α,β-monoethylenically unsaturated amino esters, for instance 2-(dimethylamino)ethyl acrylate (ADAM), 2-(dimethylamino)ethyl methacrylate (DMAM), 3-(dimethyl-amino)propyl methacrylate, 2-(tert-butylamino)ethyl methacrylate, 2-(dipentylamino)ethyl methacrylate, 2-(diethylamino)ethyl methacrylate,
vinylpyridines,
vinylamine,
vinylimidazolines,
monomers that are precursors of amine functions such as N-vinylformamide, N-vinyl-acetamide, etc., which generate primary amine functions by simple acidic or basic hydrolysis.

As examples of cationic monomers A_{C} from which the units B may be derived, mention may be made of:
ammoniumacryloyl or acryloyloxy monomers such as
   trimethylammoniumpropylmethacrylate chloride,
   trimethylammoniumethylacrylamide or methacrylamide chloride or bromide,
   trimethylammoniumbutylacrylamide or methecrylamide methyl sulfate,
   trimethylammoniumpropylmethacrylamide methyl sulfate (MES),
   (3-methacrylamidopropyl)trimethylammonium chloride (MAPTAC).
   (3-acrylamidopropyl)trimethylammonium chloride (APTAC),
   methacryloyloxyethyltrimethylammonium chloride or methyl sulfate,
   acryloyloxyethyltrimethylammonium chloride; or acryloyloxyethyltrimethylammonium methyl sulfate (Adamquat Cl or Adamquat MeS),
   methyldiethylammonium ethyl acrylate methyl sulfate (Adaequat MeS),
   benzyldimethylammonium ethyl acrylate chloride or methyl sulfate (Adamquat BZ 80),
   1-ethyl-2-vinylpyridinium or 1-ethyl-4-vinylpyridinium bromide, chloride or methyl sulfate;
   N,N-dialkyldiallylamine monomers, such as N,N-dimethyldiallylammonium chloride (DADMAC);
   dimethylaminopropylmethacrylamide N-(3-chloro-2-hydroxypropyl)trimethylammonium chloride (DIQUAT chloride).
   dimethylaminopropylmethacrylamide N-(3-methyl sulfate-2-hydroxypropyl)trimethyl-ammonium methyl sulfate (DIQUAT methyl sulfate);
   the monomer of formula

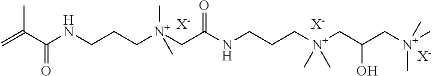

n which X⁻ is an anion, preferably chloride or methyl sulfate.

As examples of potentially cationic monomers A_{C} from which the units A_{C} may be derived:
α,β-monoethylenically unsaturated carboxylic acid N,N-(dialkylamino-ω-alkyl)amides such as N,N-dimethylaminomethyl-acrylamide or -methacrylamide, 2-(N,N-dimethylamino)ethyl-acrylamide or -methacrylamide, 3-(N,N-dimethylamino)propyl-acrylamide or
methacrylamide, 4-(N,N-dimethylamino)butyl-acrylamide or -methacrylamide,
α,β-monoethylenically unsaturated amino esters, such as 2-(dimethylamino)ethyl acrylate (ADAM), 2-(dimethylamino)ethyl methacrylate (DMAM), 3-(dimethylamino)propyl methacrylate, 2-(tert-butylamino)ethyl methacrylate, 2-(dipentylamino)ethyl methacrylate; 2-(diethylamino)ethyl methacrylate,
vinylpyridines,
vinylamine,
vinylimidazolines,
monomers that are precursors of amine functions such as N-vinylformamide, N-vinylacetamide, etc., which generate primary amine functions by simple acidic or basic hydrolysis,

### Units A_{A}

As examples of anionic or potentially anionic monomers A_{A} from which anionic or potentially anionic units A_{A} may be derived, mention may be made of;
monomers bearing at least one carboxylic function, such as α,β-ethylenically unsaturated carboxylic acids or the corresponding anhydrides, such as acrylic, methacrylic or maleic acids or anhydrides, fumaric acid, itaconic acid, N-methacryloylalanine, N-acryloylglycine and water-soluble salts thereof,
monomers that are precursors of carboxylate functions, such as tent-butyl acrylate, which generate, after polymerization, carboxylic functions by hydrolysis,
monomers bearing at least one sulfate or sulfonate function or a corresponding acid function, such as 2-sulfooxyethyl methacrylate, vinylbenzenesulfonic acid, allylsulfonic acid, 2-acrylamide-2-methylpropanesulfonic, sulfoethyl acrylate or methacrylate, sulfopropyl acrylate or methacrylate, and water-soluble salts thereof,
monomers bearing at least one phosphonate or phosphate function or a corresponding acid function, for instance vinylphosphonic acid, etc., ethylenically unsaturated phosphate esters such as phosphates derived from hydroxyethyl methacrylate (Empicryl 6835 from Rhodia) and those derived from polyoxyalkylene methacrylates and water-soluble salts thereof.

### Units B

The units B are hydrophobic and/or amphiphilic units, which may constitute the groups B. They are derived from monomers B. They may thus be amphiphilic units Bₐₘₚₕᵢ, hydrophobic units B_{phobic}, or a mixture or combination of such units. The units Bₐₘₚₕᵢ are derived from amphiphilic monomers Bₐₘₚₕᵢ, and the units B_{phobic} are derived from monomers B_{phobic}.

Amphiphilic monomers are known to those skilled in the art. They have a polymerizable part, a hydrophilic part, and one or more hydrophobic part(s), The polymerizable part is generally an ethylenically unsaturated group. The hydrophilic part generally comprises poly(ethoxy and/or propoxy) units, preferably polyethoxy, with an average number of ethoxy and/or propoxy units preferably greater than 2, preferably 5, for example greater than 10. If propoxy and ethoxy groups are present, they may be arranged in statistical form or in block form The hydrophobic part may be a hydrocarbon-based group comprising at least 3 carbon atoms, for example an alkyl, arylalkyl, alkaryl, arylalkylaryl or (polyarylalkyl)aryl or terphenyl group. The monomer may especially contain bonding groups between the various parts, especially a group —O—, or —COO—, or —CONH— or at least one urethane group (including groups derived from isocyanates, especially groups generated from aromatic isocyanates such as TDI). Useful amphiphilic monomers are monomers often referred to as surfactant monomers.

As examples of amphiphilic monomers Bₐₘₚₕᵢ from which units Bₐₘₚₕᵢ may be derived, mention may be made of:
poly(ethoxylated and/or propoxylated) C₃-C₃₀ aliphatic alcohol acrylates or methacrylates, the aliphatic part of which is, where appropriate, substituted with one or more hydroxyls preferably at the end of the aliphatic group, for example Sipomer BEM from Rhodia (polyoxyethylene ω-behenyl methacrylate optionally as a mixture), Sipomer HPM100 from Rhodia, and PLEX6877-O,
poly(ethoxylated and/or propoxylated) polystyrylphenol acrylates or methacrylates, for example Sipomer SEM-25 from Rhodia (polyoxyethylene ω-tristyrylphenyl methacrylate), poly(ethoxylated and/or propoxylated) alkylphenol acrylates or methacrylates,

As examples of hydrophobic monomes B_{phobic} from which units B_{phobic} may be derived, mention may be made of:
vinylaromatic monomers such as styrene, α-methylstyrene, para-chloromethylstyrene. vinyltoluene, etc.,
vinyl or vinylidene halides, such as vinyl chloride or vinylidene chloride,
C₁-C₃₀ and preferably C₄-C₂₂ alkyl esters of α,β-monoethylenically unsaturated acids such as methyl, ethyl, butyl, 2-ethylhexyl, isooctyl, lauryl, isodecyl or stearyl acrylates and methacrylates,
vinyl or allyl alcohol esters of saturated carboxylic acids such as vinyl or allyl acetates, propionates, versatates, stearates, etc.,
α,β-monoethylenically unsaturated nitriles containing from 3 to 12 carbon atoms, such as acrylonitrile, methacrylonitrile, etc.,
α-olefins, such as ethylene, propylene etc.,
conjugated dimer, such as butadiene, isoprene or chloroprene,
mixtures or combinations thereof.

### Composition of the Copolymer c)

The copolymer of the invention may have a mole ratio between the sum of the units A_{Z} and Aₒₜₕₑᵣ and the units B of between 1/99 and 99,9/0.1, preferably between 80/20 and 99.5/0.5 and preferably between 95/5 and 99.5/0.5,

The mole ratios and the proportions of each type of unit, chain or group may especially be likened to the mole ratios and proportions of the monomers used to prepare the units, chains or groups.

Surprisingly, the copolymer affords significant effects on the rheology even at low contents of units B.

According to one particular embodiment, the copolymer comprises only units A and B. For example, the macromolecular chain A comprises only units A_{Z}. According to one particular embodiment, the copolymer comprises units A, B and A_{A} and/or A_{C}, but substantially does not comprise any units A_{N} (i.e. not more than 1 mol %, preferably not more than 0.5 mol %, preferably not more than 0.1 mol %, and even 0%). For example, the macromolecular chain A comprises units A_{Z} and units A_{A} and/or A_{C}, but substantially does not comprise any units A_{N} (i.e. not more than 1 mol %, preferably not more than 0.5 mol %, preferably not more than 0.1 mol %, and even 0%).

According to one particular embodiment, the copolymer comprises units A, B and Aₒₜₕₑᵣ, for example units A_{A} and/or A_{C} and/or A_{N}, especially units A_{Z} and A_{N}, in a mole ratio between the units A7 and the total of the units Aₒₜₕₑᵣ of between 99/1 and 1/99, for example between 90/1 and 10/90. This ratio may especially be between 99/1 and 90/10, or between 90/10 and 80/20, or between 80/20 and 70/30, or between 70/30 and 60/40, or between 60/50 and 50/50, or between 50/50 and 40/60, or between 40/60 and 30/70, or between 30/70 and 20/80, or between 20/80 and 10/90, or between 10/90 and 1/99. For example, the macromolecular chain A comprises units A_{Z} and units Aₒₜₕₑᵣ, for example units A_{A} and/or A_{C} and/or A_{N}, especially units A_{Z} and A_{N}, with a mole ratio between the units A_{z} and the total of the units Aₒₜₕₑᵣ of between 99/1 and 1/99, for example between 90/1 and 10/90. This ratio may especially be between 99/1 and 90/10, or between 90/10 and 80/20, or between 80/20 and 70/30, or between 70/30 and 60/40, or between 60/50 and 50/50, or between 50/50 and 40/60, or between 40/60 and 30/70, or between 30/70 and 20/80, or between 20/80 and 10/90, or between 10/90 and 1/99,

The molar mass of the copolymer may, for example, be between 100 000 and 10 000 000 g/mol, preferably between 200 000 and 5 000 000 g/mol, for example between 500 000 and 3 000 000 or 4 000 000 g/mol. The polydispersity index may be relatively high, for example greater than 3, or even 4 for copolymers of relatively high masses.

The average molar mass of a segment of macromolecular chain A (between two groups B) may, for example, be greater than 50 000 g/mol and preferably 100 000 g/mol.

It is mentioned that the copolymer may be in any practical form, for example in solid or dry form or in vectorized form, for example in the form of a solution, an emulsion or a suspension, especially in the form of an aqueous solution. The vectorized form, for example an aqueous solution, may especially comprise from 5% to 50% by weight of the copolymer, for example from 10% to 30% by weight. The aqueous solution may especially be a solution obtained via a process of preparation in aqueous phase, especially a controlled micelle polymerization process. It may comprise some of the compounds used in the preparation process, especially a surfactant, generally in modest amount.

### Process for Preparing the Copolymer c)

The copolymer c) may be prepared via any suitable copolymerization process. It may especially be a radical polymerization process. It may be a micelle polymerization process.

The process may constitute an alternative description of the copolymers, and there is therefore no absolute attachment to the description of the copolymers described above, especially as regards their architecture (the ways in which the various units are distributed or arranged). Everything indicated above regarding the nature, the amounts and the ratios of the units that may be present in the copolymer, or as regards the nature, amounts and ratios of the monomers from which they may be derived, may be applied to the process of the invention, and will not always be repeated hereinbelow. It is noted that if monomers A_{Z precursor} are used, the mole amounts and ratios given for the units A_{Z} may be applied during the process. That which was indicated regarding the arrangement of the units in the copolymer of the invention may be optionally applied to the process of the invention and will not always be repeated hereinbelow. That which was indicated regarding the molecular masses of or in the copolymer of the invention may be optionally applied to the process of the invention and will not always be repeated hereinbelow.

Thus, a suitable process is a process comprising the following steps:
a) an aqueous fluid B is prepared
   the aqueous fluid B comprising:
      at least one ethylenically unsaturated monomer B, which is hydrophobic (B_{phobic}) and/or amphiphilic (Bₐₘₚₕᵢ), and
      optionally a surfactant,
      on condition that if the monomer B is a hydrophobic monomer B_{phobic}, then the aqueous fluid B comprises a surfactant,
   the monomer B being included in micelles of surfactant and/or of amphiphilic monomer Bₐₘₚₕᵢ,
b) polymerization is performed by placing in contact in aqueous medium:
   micelles of the aqueous fluid B,
   monomers A_{Z} or A_{Z precursor} and optionally other hydrophilic monomers Aₒₜₕₑᵣ, these monomers being in solution in the aqueous medium, the monomers A_{Z} being monomers comprising a betaine group, the monomers A_{Z precursor} being monomers comprising a group that can be used to form betaine groups A_{Z} via a reaction subsequent to the polymerization, and
   a source of free radicals, and
c) optionally, if monomers A_{Z precursor} have been used during step b), the units derived from these monomers are reacted so as to form betaine groups A_{Z}.

According to particular modes
the number n_{H} of monomers in the micelles is
   less than 2.6 or
   greater than or equal to 2,6,
   n_{H} preferably being between 2 and 20 and preferably between 4 and 15.
the mole ratio between the surfactant and the monomer B is
   less than or equal to 28, preferably less than or equal to 20, preferably less than or equal to 15, preferably less than or equal to 10, preferably less than or equal to 7, or
   greater than 28 and/or
the eight ratio between the surfactant, if it is present, and the monomer B is
   less than or equal to 30, preferably less than or equal to 20, preferably less than or equal to 15, preferably less than or equal to 10, preferably less than or equal to 7, or
   greater than 30.

Such a process is a process of controlled micelle polymerization type. The presence of micelles may be determined in a manner known to those skilled in the art.

It is noted that, in the process, if amphiphilic monomers B (Bₐₘₚₕᵢ) are used in the absence of surfactant, then they may be considered as both monomers and surfactants, the surfactant/monomer ratios then being considered as being equal to 1 (if the monomer Bₐₘₚₕᵢ is used in the absence of surfactant), or less than 1 (if the monomer Bₐₘₚₕᵢ is used in the presence of surfactant).

Controlled micelle polymerization processes are known to those skilled in the art. In particular, the polymerization of step b) may be performed in any manner known to those skilled in the art. It is possible in particular to vary the source of free radicals, the amount of free radicals, the phases of introduction of the various compounds and fluids, the polymerization temperature, and other parameters or operating conditions in a known and appropriate manner. A few details or indications are given hereinbelow.

According to one particular embodiment, the process comprises a polymerization of precursors of the units A_{Z} followed by a step c) of post-polymerization modification. Such processes are known to those skilled in the art. Some have been mentioned above, in the section that concerns the units A_{Z}.

It is thought that, during the controlled micelle polymerization process, a radical polymerization of the water-soluble monomers (monomers A) takes place in the aqueous phase, forming macromolecular chains comprising units derived from the water-soluble monomers A, bearing propagating free radicals at the end of the chain. It is thought that these free radicals encounter, in a random and/or statistical manner, the micelles and that the polymerization reaction then continues with the monomers of the micelle, and then continues thereafter with the water-soluble monomers of the aqueous phase. It is thought that when the polymerization reaches the micelle, it statistically polymerizes all or some of the monomers of the micelles before repropagating in the aqueous phase, thus forming groups of units derived from the monomers included in the micelles (monomers B) within macromolecular chains A of the water-soluble monomers. It is thus thought that the number of units in the groups is substantially equal (or within a margin of ±25% by number, or even ±10% by number), on average, to the number of monomers included in the micelle. Thus, it is thought that if a large number of monomers is included in the micelle, then the groups will comprise a large number of units. It is thought and has been found, especially, surprisingly, that this has a significant influence on the properties of the copolymers. The size of the micelles of a surfactant and thus the capacity of the micelles to include more or less large amounts of hydrophobic monomers is especially linked to the amount of surfactant. It is thought and has been found, especially, surprisingly, that the smaller the surfactant/monomer B ratio, the larger the amount of monomer B included in the micelles, and/or the larger the number of units B in groups B, and/or the more the copolymer shows advantageous effects in terms of rheological properties. This may especially be translated in terms of process and/or structure by the number n_{H} defined above. It has especially been found that the use of amphiphilic monomers B, and/or the presence of amphiphilic units B, affords advantageous effects in terms of rheological properties. It is mentioned that if amphiphilic monomers B are used, they may have a contribution in the formation of a micelle. If no surfactant is associated therewith, they may auto-form micelles. If a surfactant is combined therewith, they may participate in the micellization (co-micellization with the surfactant) and/or may simply enter the micelle. When amphiphilic monomers B (Bₐₘₚₕᵢ) are used, a number n_{H} may be determined by evaluating the aggregation number and the critical micelle concentration of the association of the surfactant and of monomer Bₐₘₚₕᵢ via literature techniques. Preferably, in the embodiment in which amphiphilic monomers B are used and/or when units Bₐₘₚₕᵢ are present in the copolymer; the conditions relating to the number n_{H} with these monomers or units, alone or combined with a surfactant; are verified (by counting the amphiphilic monomer as surfactant and also as monomer).

The process may be a process of batch type, of semi-batch type or even of continuous type. A process of semi-batch type typically comprises a phase of gradual introduction of at least one monomer (comonomer), preferably of all the monomers (comonomers), into a reactor, without continuous extraction of the reaction product, the reaction product, comprising the polymer, being recovered in a single stage at the end of reaction.

Step b) may be performed in batch, semi-batch or even continuous manner. Step a) may be performed in batch, semi-batch or even continuous manner. If step b) is of semi-batch and/or continuous type, then step a) may be performed in batch manner (with storage); in semi-batch manner (where appropriate with storage phases before introduction into the polymerization medium) or in continuous manner (preparation followed directly by introduction into the polymerization medium).

The process may especially be performed in one of the following manners:
batch step a), and
batch step b),
   or
continuous or semi-batch step a), and
semi-batch step b),
   or
batch step a), and
semi-batch step b).

The processes in which step b is performed in semi-batch manner, in particular with a batch step a), prove to be particularly efficient and suitable. They especially make it possible to improve the regularity of the composition of the copolymer and/or to avoid composition derivatives, especially at the end of reaction.

For example, step b) may comprise the following steps:
b1) an aqueous solution A comprising the monomers A_{Z} or A_{Z precursor}, and optionally a salt is prepared,
b2) at least part of the aqueous solution A, at least part of the aqueous fluid B and at least part of the source of free radicals are placed in contact, to form a reaction mixture,
b3) polymerization is performed by generating free radicals in the reaction mixture, preferably by heating,
b4) where appropriate, the remaining parts of the aqueous solution A and/or of the aqueous fluid B, and/or of the source of free radicals are added to the reaction mixture, each possibly being added in a single stage, or continuously. In the present patent application, the term “continuously” covers gradual introductions (uniform sporadic introductions over a given time) or progressive introductions (progressive introduction without stoppage over a given time),

It should be noted that some of the steps among step a) or steps b1), b2) and b3) may be performed simultaneously. Thus, the polymerization of step b3) continues during step b4), if there is one. It is indicated that steps a) and b1) may be performed simultaneously, separately.

Steps b2), b3) and b4) may be performed in a device known as a reactor.

During step b2), it is especially possible to introduce all of the aqueous solution A and/or all of the aqueous fluid B, and/or all of the source of free radicals. It is preferred not to introduce all of the aqueous fluid B, and to introduce it continuously. It is preferred not to introduce all of the aqueous solution A, and to introduce it continuously.

During step b2), a source of free radicals is placed in contact with at least some of the monomers of the aqueous solution A and at least some of the aqueous fluid B. The source of free radicals (all or some) may have been introduced beforehand into the aqueous solution A and/or into the aqueous fluid B. Alternatively, the source of free radicals (all or some) may have been introduced into the reactor in which the aqueous solution A and the aqueous fluid B are placed in contact, independently of the aqueous solution A (all or some) and of the aqueous fluid B (all or some), for example during the constitution of a tank stock.

It is noted that the aqueous solution A and the aqueous fluid B may be premixed before being placed in contact with the source of free radicals. It is especially possible to prepare separately the aqueous fluid B and the aqueous solution A, and then to mix them together. However, it is not excluded to add the monomers A to the aqueous fluid B or to mix all of the ingredients of the premix together (in this case, the aqueous fluid B and the aqueous solution A are combined). According to one embodiment, some of the premix may be placed in contact with the source of free radicals (all or some) during step b2) and the rest of the premix may be introduced during a step b4).

A few sequences that may be performed are detailed below.

According to a sequence of batch type, the process may be performed in the following manner:
i) the aqueous fluid B is prepared (step a)
ii) the aqueous solution A is prepared (step b1)
iii) the aqueous fluid B and the aqueous solution A are mixed together and the whole amount is introduced into the reactor (the mixing may be performed in the reactor)
iv) the source of free radicals is added, preferably a partially soluble initiator (step b2)
v) polymerization is performed with heating (step b3).

It is noted that heating may be begun before adding the source of free radicals,

According to another sequence of batch type, the process may be performed in the following manner:
i) a feed stock comprising the source of free radicals, preferably with water, is prepared in the reactor,
ii) the aqueous fluid B is prepared (step a)
iii) the aqueous solution A is prepared (step b1)
iv) the aqueous fluid B and the aqueous solution A are mixed together and the whole is introduced into the reactor (the mixing may be performed in the reactor) (step b2)
iv) polymerization is performed with heating.

According to a sequence of semi-batch type, the process may be performed in the following manner:
i) the aqueous fluid B is prepared (step a)
ii) the aqueous solution A is prepared (step b1)
iii) the aqueous fluid B and the aqueous solution A are mixed together to obtain a premix (alternatively, the premix may be prepared directly, by omitting step i) and/or step ii))
iv) a feed stock comprising part of the premix, and the source of free radicals, is prepared in the reactor, preferably with water
v) the polymerization is initiated by heating
vi) the rest of the premix is added continuously, with heating, so as to continue the polymerization,
the source of free radicals being introduced into the premix (step or separately, in total or in part, into the feed stock (step iv), the rest optionally being introduced during step vi).
It is noted that, alternatively to premixing, the aqueous solution A and the aqueous fluid B may be introduced separately (it being especially possible for the source of free radicals then to be introduced into the aqueous fluid B and/or into the aqueous solution A, if the latter is introduced into the premix)

According to another sequence of semi-batch type, the process may be performed in the following manner:
i) the aqueous fluid B is prepared (step a)
ii) the aqueous solution A is prepared (step b1)
iii) the aqueous fluid B and the aqueous solution A are mixed together to obtain a premix (it is alternatively possible to prepare the premix directly, by omitting step i) and/or step ii))
iv) a feed stock comprising water, and optionally surfactant (the latter may contribute toward conserving the micelles of the aqueous fluid B during the first addition to the reactor) is prepared in the reactor
v) the premix is added continuously with heating, so as to polymerize, the source of free radicals being introduced into the premix, or separately, in total or in part, into the feed stock (step iv), the rest being optionally introduced during step v).
It is noted that heating may be begun before the introduction of the premix.
It is noted that, alternatively to premixing, the aqueous solution A and the aqueous fluid B may be introduced separately (it being possible especially for the source of free radicals then to be introduced into the aqueous fluid B and/or into the aqueous solution A, if the latter is introduced into the premix)
   Surfactants Optionally used for the Preparation of the Copolymer c)

Any surfactant capable of forming micelles in water may be used. The use of surfactant is particularly useful for the formation of micelles if the monomers B are solely hydrophobic monomers B_{phobic}. The surfactant is generally used, in particular for polymerization in the absence of monomers Bₐₘₚₕᵢ, at a concentration above the critical micelle concentration. It is especially possible to use at least one anionic, nonionic, amphoteric (including zwitterionic) or cationic surfactant, or a mixture or combination thereof. Anionic or nonionic surfactants may preferably be used.

It is especially possible to use standard anionic surfactants chosen especially from alkyl sulfates, such as sodium lauryl sulfate, alkylsulfonates, alkylaryl sulfates, alkylarylsulfonates such as sodium dodecylbenzenesulfonate, aryl sulfates, arylsulfonates, ethoxylated alkyls, ethoxylated alkylaryls, sulfated or phosphated ethoxylated alkyls or ethoxylated alkylaryls, or salts thereof, sulfosuccinates, alkali metal alkyl phosphates, hydrogenated or non-hydrogenated abietic acid salts, or fatty acid salts such as sodium stearate.

It is especially possible to use standard anionic surfactants chosen especially from ethoxylated and/or propoxylated alcohols, ethoxylated and/or propoxylated fatty acids, block copolymers of polyethylene oxide and of polypropylene oxide, etc.

It is noted that the reaction medium may especially comprise an organic or inorganic salt. This salt may be introduced, for example, into the aqueous solution A. The salt may facilitate the maintenance in solution of the copolymer obtained, in particular if it has a high molar mass, or may improve the maintenance and/or introduction of the monomers B (in particular B_{phobic}) in the micelles. As salts that may be used, mention is made especially of salts whose cation is an alkali metal, an alkaline-earth metal or an ammonium (for example NH₄⁺) and whose anion is a halogen, a phosphate, a sulfate or a nitrogen oxide. Mention is made, for example, of sodium chloride, sodium sulfate or ammonium sulfate.

### Polymerization

Any source of free radicals may be used. It is especially possible to generate free radicals spontaneously, for example by raising the temperature, with suitable monomers such as styrene (monomer B). Free radicals may be generated by irradiation, especially by UV irradiation, preferably in the presence of suitable UV-sensitive initiators. Initiators (or “primers”) or initiator systems, of radical or redox type, may be used. The source of free radicals may or may not be water-soluble. Primers that are water-soluble, or at least partially water-soluble (for example water-soluble to at least 50% by weight) may preferably be used.

In general, the larger the amount of free radicals, the more easily the polymerization is initiated (it is promoted), but the lower the molecular masses of the copolymers obtained,

The following initiators may especially be used:
hydrogen peroxides such as: tert-butyl hydroperoxide, cumene hydroperoxide.

t-butyl peroxyacetate, t-butyl peroxybenzoate, t-butyl peroxyoctoate, t-butyl peroxyneodecanoate, t-butyl peroxyisobutarate, lauroyl peroxide, t-amyl peroxypivalate, t-butyl peroxypivalate, dicumyl peroxide, benzoyl peroxide, potassium persulfate, ammonium persulfate,
azo compounds such as: 2,2′-azobis(isobutyronitrile), 2,2′-azobis(2-butanenitrile), 4,4′-azobis(4-pentanoic acid), 1,1′-azobis(cyclohexanecarbonitrile), 2-(t-butylazo)-2-cyanopropane, 2,2′-azobis[2-methyl-N-(1,1)-bis(hydroxymethyl)-2-hydroxyethyl]propionamide, 2,2′-azobis(2-methyl-N-hydroxyethyl]propionamide, 2,2″-azobis(N,N′-dimethyleneisobutyramidine)dichloride, 2,2′-azobis(2-amidinopropane)dichloride, 2,2′-azobis(N,N′-dimethyleneisobutyramide), 2,2′-azobis(2-methyl-N-[1,1-bis(hydroxymethyl)-2-hydroxyethyl]propionamide), 2,2′-azobis(2-methyl-N-[1,1-bis(hydroxymethyl)ethyl]-propionamide), 2,2′-azobis[2-methyl-N-(2-hydroxyethyl)propionamide], 2,2′-azobis(isobutyramide)dihydrate,
redox systems comprising combinations such as:
mixtures of hydrogen peroxide, of alkyl, peresters, percarbonates and the like and of any iron salts, titanium salts, zinc formaldehyde sulfoxylate or sodium formaldehyde sulfoxylate, and reducing sugars,
alkali metal or ammonium persulfates, perborate or perchlorate in combination with an alkali metal bisulfite, such as sodium metabisulfite, and reducing sugars, and
alkali metal persulfates in combination with an arylphosphinic acid such as benzenephosphonic acid and the like, and reducing sugars.

The polymerization temperature may especially be between 25° C. and 95° C. The temperature may depend on the source of free radicals. If it is not a source of UV initiator type, it will be preferred to perform the process between 50° C. and 95° C. and more preferably between 60° C. and 80° C. In general, the higher the temperature, the more easily the polymerization is initiated (it is promoted), but the smaller the molecular masses of the copolymers obtained.

### Cosmetic Composition

The cosmetic composition is an aqueous cosmetic composition comprising a) a surfactant system, b) optionally at least one salt, and the copolymer c). It may comprise other ingredients,

It is mentioned that the cosmetic composition is an aqueous composition. It thus comprises water as cosmetically acceptable vector. It is not excluded to add to the water other cosmetically acceptable vectors, such as ethanol.

The cosmetic composition may especially be a composition for cleansing the skin and/or the hair. It may especially be a rinse-out composition. It may especially be a foaming composition. It may especially be a shampoo, a hair conditioner (intended to be rinsed out), a shower gel, a cleansing product for personal hygiene, a face cleanser, an exfoliant gel, a liquid hand cleanser, a hair coloring product. It may also be a leave-in composition, in particular a haircare composition, for example a leave-in hair conditioner, a disentangling milk, a disentangling lotion, a smoothing lotion, a cuticle coating, a styling and/or restyling haircare product. It may also be an antisun product, a care cream, a makeup-removing product, a makeup, a makeup-removing or moisturizing wipe lotion, a shaving foam, a styling or fixing mousse, or a styling or fixing gel.

For rinse-out hair conditioners, the composition may be a relatively viscous formulation, for example a cream, in the form of an emulsion.

According to advantageous embodiments, the composition is a skincare and/or haircare composition, in the form of a fluid or in another form, preferably for treating and/or protecting and/or modifying the appearance of the skin and/or the hair, intended to be left on the skin and/or the hair after application.

The surfactant system may be formed from a single surfactant or from a mixture or combination of surfactants. Any surfactant included in the composition is considered as forming part of the surfactant system. The structuring agent(s), whether or not they are surfactants, are considered as forming part of the surfactant system. Surfactants and structuring agents are known to those skilled in the art. It is mentioned that surfactants are generally molecules not containing a macromolecular chain, optionally apart from polyoxyalkylene chains (for example polyoxyethylene and/or polyoxypropylene). They may typically have molar masses of less than 1000 g/mol or 500 g/mol, This is likewise the case for the structuring agents. It is mentioned that the structuring agents in the present patent application do not cover polymers containing macromolecular chains other than polyoxyalkylene chains.

The surfactant system may especially comprise an anionic, cationic, amphoteric (true amphoteric or zwitterionic) or nonionic surfactant, or a mixture or combination of such surfactants. It may comprise an anionic surfactant and/or a nonionic surfactant.

According to one embodiment, the surfactant system comprises at least one anionic surfactant, the anionic surfactant(s) preferably constituting at least 50% by weight of the total weight of surfactant(s) present in the system. According to one particularly advantageous embodiment, especially the embodiment including at least 50% by weight of anionic surfactant(s), the surfactant system comprises at least one anionic surfactant and at least one amphoteric surfactant (true amphoteric or zwitterionic),

According to one embodiment, the surfactant system comprises at least one nonionic surfactant, the nonionic surfactant(s) preferably constituting at least 50% by weight of the total weight of surfactant(s) present in the system,

Surfactants and/or structuring agents that may be used are detailed hereinbelow.

According to one particular embodiment, the surfactant system (i.e. the compounds of the system and the relative proportions thereof) and the optional salt, and the amounts thereof are such that the composition has organized phases of surfactant(s). They may especially be organized phases of surfactant(s) of giant micelle type

In one particular variant:
the surfactant system comprises an anionic surfactant,
the composition comprises a salt and/or a structuring agent.

In one particular variant:
the surfactant system comprises an anionic surfactant,
the composition comprises a salt and/or a structuring agent, and
the composition has organized phases of surfactant(s) of giant micelle type.

The anionic surfactants may especially be chosen from the following surfactants:
alkyl ester sulfonates, for example of formula R—CH(SO₃M)-CH₂COOR′, or alkyl ester sulfates, for example of formula R—CH(OSO₃M)—CH₂COOR′, in which R represents a C₈-C₂₀ and preferably C₁₀-C₁₆ alkyl radical, R′ a C₁-C₆ and preferably C₁-C₃ alkyl radical and M an alkaline-earth metal cation, for example sodium, or an ammonium cation. Mention may be made most particularly of methyl ester sulfonates whose radical R is of C₁₄-C₆;
alkylbenzenesulfonates, more particularly of C₉-C₂₀, primary or secondary alkylsulfonates, especially of C₈-C₂₂, and alkylglyceryl sulfonates;
alkyl sulfates, for example of formula ROSO₃M, in which R represents a C₁₀-C₂₄ and preferably C₁₂-C₂₀ alkyl or hydroxyalkyl radical; M represents a cation of the same definition as above;
alkyl ether sulfates, for example of formula RO(OA)ₙSO₃M in which R represents a C₁₀-C₂₄ and preferably C₁₂-C₂₀ alkyl or hydroxyalkyl radical; OA representing an ethoxylated and/or propoxylated group; M representing a cation of the same definition as above, n generally ranging from 1 to 4, for instance lauryl ether sulfate with n=2;
alkylamide sulfates, for example of formula RCONHR′OSO₃M in which R represents a C₂-C₂₂ and preferably C₆-C₂₀ alkyl radical, R represents a C₂-C₃ alkyl radical, M representing a cation of the same definition as above, and also the polyalkoxylated (ethoxylated and/or propoxylated) derivatives thereof (alkylamido ether sulfates);
saturated or unsaturated fatty acid salts, for example those of C₈-C₂₄ and preferably of C₁₄-C₂₀ and of an alkaline-earth metal cation, N-acyl N-alkyltaurates, alkylisethionates, alkylsuccinamates and alkylsulfosuccinates, alkylglutamates, sulfosuccinate monoesters or diesters, N-acyl sarcosinates and polyethoxycarboxylates;
phosphate monoesters and diesters, for example having the following formula: (RO)ₓ—P(═O)(OM), in which R represents an alkyl, alkylaryl, arylalkyl or aryl radical, which are optionally polyalkoxylated, x and x′ being equal to 1 or 2, on condition that the sum of x and x′ is equal to 3, M representing an alkaline-earth metal cation.

The nonionic surfactants may especially be chosen from the following surfactants:
alkoxylated fatty alcohols: for example laureth-2, laureth-4, laureth-7, oleth-20
alkoxylated triglycerides
alkoxylated fatty acids
alkoxylated sorbitan esters
alkoxylated fatty amines
alkoxylated bis(1-phenylethyl)phenols
alkoxylated tris(1-phenylethyl)phenols
alkoxylated alkylphenols
products resulting from the condensation of ethylene oxide with a hydrophobic compound resulting from the condensation of propylene oxide with propylene glycol, such as the Pluronic products sold by BASF;
products resulting from the condensation of ethylene oxide with the compound resulting from the condensation of propylene oxide with ethylenediamine, such as the Tetronic products sold by BASF;
alkylpolyglycosides, for instance those described in U.S. Pat. No. 4,565,647 or alkyl glucosides;
fatty acid amides, for example of C₈-C₂₀, especially fatty acid monoalkanolamides, for example MEA cocamide or MIPA cocamide.

The amphoteric surfactants (true amphoteric surfactants comprising an ionic group and a potentially ionic group of opposite charge, or zwitterionic surfactants simultaneously comprising two opposite charges) may especially be chosen from the following surfactants:
betaines in general, especially carboxy betaines, for example lauryl betaine (Mirataine BB from the company Rhodia) or octyl betaine or cocobetaine (Mirataine BB-FLA from Rhodia); amidoalkyl betaines, for instance cocamidopropyl betaine (CAPB) (Mirataine BDJ from the company Rhodia or Mirataine BET C-30 from Rhodia);
sulfobetaines or sultaines, for instance cocamidopropyl hydroxy sultaine (Mirataine CBS from the company Rhodia);
alkylamphoacetates and alkylamphodiacetates, for instance those comprising a coco or lauryl chain (Miranol C2M Conc NP, C32 and L32 especially, from the company Rhodia);
alkylamphopropionates or alkylamphodipropionates (Miranol C2M SF);
alkyl amphohydroxypropyl sultaines (Miranol CS);
alkylamine oxides, for example lauramine oxide (INCI).

The cationic surfactants may especially be chosen from primary, secondary or tertiary, optionally polyethoxylated fatty amine salts, quaternary ammonium salts such as tetraalkylammonium, alkylamidoalkylammonium, trialkylbenzylammonium, trialkylhydroxyalkylammonium or alkylpyridinium chlorides or bromides, imidazoline derivatives and amine oxides of cationic nature. An example of a cationic surfactant is cetrimonium chloride or bromide (INCI).

The surfactant or non-surfactant structuring agents may be chosen especially from the following compounds:
alkanolamides, especially cocamide MEA or cocamide DEA or ocamide MIPA,
optionally unsaturated and/or branched fatty acids, preferably of C₈-C₂₄, especially lauric acid, oleic acid or isostearic add,
optionally unsaturated and/or branched fatty add esters, preferably of C₈-C₂₄, especially methyl esters, especially of lauric add, of oleic acid or of isostearic acid,
optionally unsaturated and/or branched fatty acids, preferably of C₈-C₂₄, which are (poly)alkoxylated, especially (poly)ethoxylated or (poly)propoxylated, for example propylene glycol isostearate,
fatty alcohols, which are preferably sparingly ethoxylated and/or propoxylated, preferably of C₈-C₂₄, especially laureth-2, laureth-3 or laureth-4,
trihydroxystearine,
amine oxides, especially of C₈-C₂₄, especially dimethylamine oxides.

The composition may comprise, for example; from 0.1% to 3% by weight, for example from 0.5 to 2% by weight, of structuring agent(s), if present.

The amount of surfactant(s) (including the optional structuring agents) may especially be between 1% by weight and 50% by weight, preferably between 1% and 20%, preferably between 1% and less than 5% or between 5% and 20%. The copolymer c) is, surprisingly, efficient as regards rheology at relatively low contents.

The salts that may be used may especially be alkali metal or alkaline-earth metal salts, or ammonium salts. They may especially be chloride, bromide, sulfate or nitrate salts. As examples of salts that may be used, mention is made especially of:
NaCl, KCl, NH₄Cl,
—MgCl₂, MgSO₄
NaBr, KBr.

The amount of salt(s), if present, may especially be between more than 0% and 10% by weight, preferably between more than 0% and 10% by weight, for example from 0,01% to 5% by weight, for example from 0.01% to 4% by weight, for example from 0.01% to 3.5% by weight, for example from 0.01% to 3% by weight,for example from 0.01% to 2.5% by weight, for example from 0.01% to 2% by weight, for example from 0.01% to 1.5% by weight, for example from 0.01% to 1% by weight, for example from 0.01% to 0,5% by weight. The amount may especially be from 0.01% to 0.5% by weight or from 0.5% to 1%, or from 1% to 1.5%, or from 1.5% to 2%, or from 2% to 2.5%, or from 2.5% to 3% or from 3% to 3.5% or from 3.5% to 4%, or from 4% to 5% or from 5% to 10%.

As examples of useful compositions, mention may be made of:
“Sodium” compositions for shampoos typically comprising 12% to 16% by weight of sodium alkyl ether sulfate (for example sodium lauryl ether sulfate “SLES”) or a mixture of a sodium alkyl ether sulfate and of a sodium alkyl sulfate (for example sodium lauryl sulfate “SLS”), 1% to 3% of an amphoteric surfactant (for example cocoamidopropylbetaine “CAPB”), 0.5% to 2% of a salt (for example sodium chloride).
“Ammonium” compositions for shampoos typically comprising 12% to 16% by weight of ammonium alkyl ether sulfate (for example ammonium lauryl ether sulfate “ALES”) or a mixture of ammonium alkyl ether sulfate and of ammonium alkyl sulfate (for example ammonium lauryl sulfate “ALS”), 1% to 3% of an amphoteric surfactant (for example cocoamidopropylbetaine “CAPR”), 0 to 2% of a salt (for example ammonium chloride).
“Sodium” compositions for shower gels, typically comprising 6% to 10% by weight of sodium alkyl ether sulfate (for example sodium lauryl ether sulfate “SLES”) or a mixture of sodium alkyl ether sulfate and of sodium alkyl sulfate (for example sodium lauryl sulfate “SLS”), 1% to 3% of an amphoteric surfactant (for example cocoamidopropylbetaine “CAPB”), 2% to 4% of a salt (for example sodium chloride),
“Sodium” compositions for shower gels, typically comprising 6% to 10% by weight of ammonium alkyl ether sulfate (for example ammonium lauryl ether sulfate “ALES”) or a mixture of ammonium alkyl ether sulfate and of ammonium alkyl sulfate (for example ammonium lauryl sulfate “ALS”), 1% to 3% of an amphoteric surfactant (for example cocoamidopropylbetaine CA PB″), 0 to 4% of a salt (for example ammonium chloride).

The amount of copolymer c) may especially be between 0.01% by weight and 10% by weight, preferably between 0.05% and 5%, preferably between 0.1% and less than 1%. The copolymer is, surprisingly, efficient as regards rheology from relatively low contents. The larger the copolymer content, the more pronounced the effect.

According to one particular embodiment, the weight ratio between the amount of polymer c) and the amount of surfactant(s) with optional structuring agent(s) is less than 1/16=0.0625 and preferably less than 0.5/16=0.03125. This ratio may especially be greater than 0.01/8=0.0125 and preferably 0.1/8=0.125. It is observed, surprisingly, that rheology effects may be observed at low ratios, i.e. with small amounts of copolymer c),

It is thought that the copolymer c) may contribute in the phase diagram (surfactant system/salt), for a given surfactant system, to shifting toward smaller amounts of surfactant system and/or of salt, the zone of presence of organized phases of surfactant(s). It is thought that this is likewise the case for a phase diagram (surfactant(s)+optional salt/structuring agent).

### Other Ingredients

The composition may comprise ingredients other than the copolymer c), the surfactant system, and the optional salt. Such ingredients may be common and known ingredients, according to the use for which the composition is intended.

They may especially be:
active agents,
conditioning agents or emollients, especially oils or polymers,
preserving agents,
dyes, pigments,
oxidizing agents.
pH regulators,
opacifiers and/or nacreous agents,
stabilizers,
additional thickeners,
agents for destabilizing emulsions during dilution,
UV screening agents,
dispersants,
sequestrants,
humectants,
film-forming polymers.
fixing polymers,
fragrances,
exfoliants or abrasive agents such as solid particles.

It is mentioned that the pH of the composition generally depends on its purpose and its use. The pH is generally between 3.5 and 7.7. It may be greater than or equal to 4.5 and more preferably 5.5. It is, for example, between 5.5 and 7.5 and preferably between 6 and 6.5. The pH obviously depends on the compounds present in the composition. It is obviously possible to use in the composition pH regulators, acids or bases, for example citric acid or sodium, potassium or ammonium hydroxide. For compositions intended for haircare, especially for leave-in hair conditioners, which may especially comprise cationic surfactants generally in small amounts (less than 5% by weight), the pH may be relatively acidic, for example from 3.5 to 5.5.

Conditioning agents or emollients may especially constitute a dispersed organic phase, for example in the form of an emulsion, in the aqueous phase.

Compounds that may be used in the organic phase are preferably chosen from compounds whose solubility in water does not exceed 10% by weight, at 20° C.

As conditioning agents or emollients, mention may be made especially of organic oils, of animal, plant or mineral origin, synthetic oils such as silicone oils (polyorganosiloxane), and also waxes of the same types, or mixtures thereof.

Among the plant oils and derivatives thereof, mention may be made especially of: almond oil (sweet almond oil), anhydrous lanolin oil, apricot kernel oil, avocado oil, castor oil, jojoba oil, olive oil, groundnut oil, sesame oil, sunflower oil, corn oil, cottonseed oil, hydrogenated legume oils, soybean oil, sulfonated castor oil, coconut oil, cocoa butter, wheatgerm oil, aloe vera, grapeseed oil, hazelnut oil, macadamia nut oil, St-Jean protuberance oil, walnut oil, hazelnut oil, borage oil, peach stone oil, virgin coconut oil, baobab oil, avocado butter, palm oil, palm kernel oil, linseed oil, coconut oil, babassu oil, wheatgerm oil.

Among the oils of animal origin, mention may be made, inter alia, of sperm whale oil, whale oil, seal oil, sardine oil, herring oil, shark oil, cod liver oil; pig fat or sheep fat (tallow).

As regards mineral oils, mention may be made, inter alia, of naphthenic or paraffinic oils (petroleum jelly, or petrolatum). Mention may also be made of perhydrosqualene and squalene.

As waxes of plant origin, mention may be made of carnauba wax.

As regards mineral oils, mention may be made, inter alia, of petroleum fractions, and naphthenic and paraffinic oils (petroleum jelly). The paraffinic waxes may similarly be suitable for preparing the emulsion.

The products derived from the alcoholysis of the abovementioned oils may also be used.

It would not constitute a departure from the context of the present invention to use, as organic phase, at least one saturated or unsaturated fatty acid, at least one saturated or unsaturated fatty alcohol, at least one fatty acid ester, or mixtures thereof.

More particularly, said acids comprise 8 to 40 carbon atoms, more particularly 10 to 40 carbon atoms, preferably 18 to 40 carbon atoms, and may comprise one or more conjugated or nonconjugated ethylenic unsaturations, and optionally one or more hydroxyl groups As regards the alcohols, they may comprise one or more hydroxyl groups.

As examples of saturated fatty acids, mention may be made of palmitic, stearic and behenic acids.

As examples of unsaturated fatty acids, mention may be made of myristoleic, palmitoleic, oleic, erucic, linoleic, linolenic, arachidonic and ricinoleic acids, and mixtures thereof.

As regards the alcohols, they more particularly comprise 4 to 40 carbon atoms, preferably 10 to 40 carbon atoms, optionally one or more conjugated or nonconjugated ethylenic unsaturations, and optionally several hydroxyl groups. Polymers comprising several hydroxyl groups may similarly be suitable, for instance polypropylene glycols.

Examples of alcohols that may be mentioned include those corresponding to the abovementioned acids.

As regards the fatty acid esters, they may advantageously be obtained from fatty acids, chosen from the compounds mentioned above. The alcohols from which these esters are prepared more particularly comprise 1 to 6 carbon atoms. Preferably, they are methyl, ethyl, propyl or isopropyl esters.

Moreover, it is not excluded to use mono-, di- and triglycerides as organic phase.

According to one embodiment, the conditioning agent or emollient, for example constituting an organic phase, is based on a polyorganosiloxane. Polyorganosiloxanes are also known as silicones. The term “silicone” or “polyorganosiloxane” means any organosiloxane compound comprising alkyl groups (for example methyl) and/or functionalized with groups other than alkyl groups.

The polyorganosiloxane is advantageously (in shampoos and hair conditioners in particular) a nonvolatile water-insoluble polyorganosiloxane. It advantageously has a viscosity of between 1000 and 2 000 000 mPa·s and preferably between 5000 and 1 000 000 mPa·s (at 25° C.). The polyorganosiloxane may especially be a polydimethylorganosiloxane (“PDMS”, INCI name: dimethicone) or a polyorganosiloxane containing amine groups (for example Amodimethicone according to the INCI name), quaternary ammonium groups (for example the silicones Quaternium 1 to 10 according to the INCI name), hydroxyl groups (terminal or nonterminal), polyoxyalkylene groups, for example polyethylene oxide and/or polypropylene oxide (as end groups, as a block in a PDMS chain, or as grafts) or aromatic groups, or several of these groups.

The polyorganosiloxanes that are useful in the cosmetics field and the characteristics thereof are known to those skilled in the art.

The polyorganosiloxanes (silicones) are preferably present in the composition or in the concentrated ingredient in emulsion form (liquid droplets of silicone dispersed in the aqueous phase). The emulsion may especially be an emulsion with a mean droplet size of greater than or equal to 2 μm, or with a mean droplet size of between 0.15 μm and 2 μm, or with a mean droplet size of less than or equal to 0.15 μm.

The droplets of the emulsion may be of more or less large size. Reference may thus be made to microemulsions, miniemulsions or macroemulsions. In the present patent application, the term “emulsion” especially covers all these types of emulsion. Without wishing to be bound to any theory, it is pointed out that microemulsions are generally thermodynamically stable systems, generally comprising large amounts of emulsifiers such as surfactants c). The other emulsions are generally systems in thermodynamically unstable state, conserving for a certain time, in metastable state, the mechanical energy supplied during the emulsification. These systems generally comprise smaller amounts of emulsifiers.

The emulsions may be obtained by mixing an outer phase, which is preferably aqueous, polyorganosiloxane, polymer for aiding deposition and, in general, an emulsifier, followed by emulsification. This process may be referred to as in-situ emulsification.

The microemulsion droplet size may be measured on an emulsion prepared prior to its introduction into the cosmetic composition, by dynamic light scattering (DQEL), for example as described below. The apparatus used consists, for example, of a Spectra-Physics 2020 laser, a Brookhaven 2030 correlator and the associated computerware. Since the sample is concentrated, it is diluted in deionized water and filtered through a 0.22 μm filter in order finally to be at 2% by weight. The diameter obtained is an apparent diameter. The measurements are taken at angles of 90° and 135°. For the size measurements, besides the standard cumulative analysis, three exploitations of the self-correlation function are used (exponential sampling or EXPSAM described by Prof. Pike, the “nonnegatively constrained least squares” or NNLS method, and the CONTIN method described by Prof. Provencher), which each give a size distribution weighted by the scattered intensity, rather than by the mass or the number. The refractive index and the viscosity of water are taken into account.

According to one useful embodiment, the composition and/or the concentrated ingredient are transparent. The composition and/or the concentrated ingredient may, for example, have a transmittance of at least 90% and preferably of at least 95%, at a wavelength of 600 nm, for example measured using a Lambda 40 UV-Vis spectrometer, at a concentration of 0.5% by weight in water.

According to another particular embodiment, the composition or the concentrated ingredient are emulsions whose mean droplet size is greater than or equal to 0.15 μm, for example greater than 0.5 μm, or 1 μm, or 2 μm, or 10 μm, or 20 μm, and preferably less than 100 μm. The droplet size may be measured on an emulsion prepared prior to its introduction into the cosmetic composition, by optical microscopy and/or laser granulometry (Horiba LA-910 laser scattering analyzer). In this embodiment, the composition and/or the concentrated ingredient preferably comprise a proportion of less than 10% by weight of emulsifier relative to the weight of polyorganosiloxane.

Among the water-soluble silicones of the composition that may be mentioned, inter alis, are dimethicone copolyols (Mirasil DMCO sold by Rhodia).

As regards silicones in the form of water-insoluble dispersions or emulsions, nonvolatile water-insoluble organopolysiloxanes may appropriately be used, among which mention may be made of polyalkylsiloxane, polyarylsiloxane, and polyalkylarylsiloxane oils, gums or resins or nonvolatile water-insoluble functionalized derivatives thereof, or mixtures thereof.

Said organopolysiloxanes are considered as being water-insoluble and nonvolatile when their solubility in water is less than 50 g/liter and their intrinsic viscosity is at least 3000 mPa·s, at 25° C.

Examples of nonvolatile water-insoluble organopolysiloxanes or silicones that may be mentioned include silicone gums, for instance the diphenyl dimethicone gum sold by the company Rhodia Chimie, and preferably polydimethylorganosiloxanes with a viscosity at least equal to 6×10⁵ mPa·s, at 25° C., and even more preferentially those with a viscosity of greater than 2×10⁶ mPa·s, at 25° C., such as Mirasil DM 500000® sold by Rhodia.

Among these low-viscosity silicones, mention may be made of cyclic volatile silicones and polydimethylorganosiloxanes of low mass.

It is also possible to use functionalized silicone derivatives, for instance amine derivatives directly in the form of emulsions or starting with a preformed microemulsion. These may be compounds known as amino silicones or hydroxyl silicones. Mention is made, for example, of the oil Rhodorsil amine 21637 (Amodimethicone) sold by the company Rhodia, and dimethiconol.

As polyorganosiloxanes that may be used mention is made especially of:

polyorganosiloxanes comprising units —Si(CH₂)₂O— and units —SiY(CH₂)O— in which Y is a —(CH₂)₃—NH(CH₂)₂—NH₂ or —(CH₂)₃—NH₂ group,

polyorganosiloxanes comprising units —Si(CH₂)₂O— and end units —HO—Si(CH₂)₂— and/or non-end units —Si(CH₂)(OH)O—,

polyorganosiloxanes comprising units —Si(CH₂)₂O— and units —SiY(CH₂)O— in which Y is -L^{X}-Z^{x}-Palk in which L^{X} is a divalent bonding group, preferably an alkyl group, Z^{X} is a covalent bond or a divalent connecting group comprising a heteroatom, Palk is a group of formula [OE]ₛ-[OP]ₜ—X′, in which OE is a group of formula —CH₂—CH₂—O—, OP is a group of formula —CH₂—CHCH₃—O— or —CHCH₃—CH₂—O—, X′ is a hydrogen atom or a hydrocarbon-based group, s is a mean number greater than 1, and t is a mean number greater than or equal to 0.

polyorganosiloxanes whose chain comprises at least one block comprising units of formula —Si(CH₂)₂O— and at least one block —[OE]ₛ-[OP]ₜ—,

polyorganosiloxanes comprising units —Si(CH₂)₂O— and/or units —Si(CH₂)RO— and/or —SiR₂O— and/or R—Si(CH₂)₂O— and/or H₃C—SiR₂O— and/or R—SiR₂O— in which R, which may be identical or different, is an alkyl group other than a methyl group, an aryl group, an alkyl group, an alkylaryl group or an aralkyl group.

The following commercially available ingredients may especially be used as polyorganosilaxanes:

Mirasil DM 500000, Rhodia (INCI: Dimethicone), for example in the form of an emulsion with a particle size of 0.6 μm or 0.9 μm,

Mirasil DME-2, Rhodia (INCI: dimethicone)

Mirasil DME30, Rhodia dimethicone)

Mirasil ADM-E, Rhodia (INCI: amodimethicone)

Dow Corning 1784 HVF, Dow Corning (INCI: Dimethiconol)

Dow Corning 1784 HMW, Dow Corning (INCI: Divinyldimethicone/dimethicone)

Mirasil DMCP-93, Rhodia (INCI: PEG/PPG-10/2 dimethicone)

Parsol SLX, DSM (INCI: Polysilicone-15)

Mirasil SM, Rhodia Simethicone)

Mirasil DMCO, Rhodia (INCI PEG/PPG-22/24 Dimethicone)

Mirasil DM 100000, Rhodia (INCI: Dimethicone)

DC200 fluid 60000, Dow Corning (INCI: Dimethicone)

DC200 fluid 300000, Dow Corning (INCI: Dimethicone).

According to particular embodiments, the silicone oils are totally or partially formed from units of formula:

R′₃₋ₐRₐSiO_{1/2} (unit M) and/or R₂SiO (unit D)

in which formulae:

a is an integer from 0 to 3;

the radicals R are identical or different and represent:
a saturated or unsaturated aliphatic hydrocarbon-based group containing from 1 to 10 carbon atoms;
an aromatic hydrocarbon-based group containing from 6 to 13 carbon atoms;
a polar organic group linked to silicon via an Si—C or Si—O—C bond;

the radicals R′ are identical or different and represent:
a saturated or unsaturated aliphatic hydrocarbon-based group containing from 1 to 10 carbon atoms;
an aromatic hydrocarbon-based group containing from 6 to 13 carbon atoms;
an —OH function;
an amino-functional or amino-functional group containing from 1 to 6 carbon atoms, linked to silicon via an Si—N bond.

Preferably, at least 80% of the radicals R represent a methyl group.

These silicones may optionally comprise preferably less than 5 mol % of units of formulae T and/or Q:

RSiO3/2 (unit T) and/or SiO₂ (unit Q)

in which formula R has the definition given above.

Examples of aliphatic or aromatic hydrocarbon-based radicals R that may be mentioned include the following groups:

alkyl, preferably optionally halogenated C₁-C₁₀ alkyl, such as methyl, ethyl, octyl or trifluoropropyl,

alkoxyalkylene, more particularly of C₂-C₁₀ and preferably of C₂-C₆, such as —CH₂—CH₂—O—CH_{3;}

alkenyls, preferably C₂-C₁₀ alkenyl, such as vinyl, allyl, hexenyl, decenyl or decadienyl;

alkenyloxyalkylene such as —(CH₂)₃—O—CH₂—CH═CH₂, or alkenyloxyalkoxyalkyl such as —(CH₂)₃—OCH₂—CH₂—O—CH═CH₂ in which the alkyl portions are preferably of C₁-C₁₀ and the alkenyl portions are preferably of C₂-C₁₀;

aryls, preferably of C₆-C₁₃, such as phenyl

Examples of polar organic groups R that may be mentioned include:

hydroxy-functional groups such as alkyl groups substituted with one or more hydroxyl or di(hydroxyalkyl)amino groups and optionally interrupted with one or more divalent hydroxyalkylamino groups. The term “alkyl” means a hydrocarbon-based chain preferably of C₁-C₁₀ and better still of C₁-C₆; examples of these groups are —(CH₂)₃—OH; —(CH₂)₄N(CH₂CH₂OH)₂: —(CH₂)₃—N(CH₂CH₂OH)—CH₂—CH₂—N(CH₂CH₂OH)₂;

amino-functional groups such as alkyl substituted with one or more amino or aminoalkylamino groups in which alkyl is as defined above; examples of these are —(CH₂)₃—NH₂; (CH₂)₃—NH—(CH₂)₂NH₂;

amido-functional groups such as alkyl substituted with one or more acylamino groups and optionally interrupted with one or more divalent alkyl-CO—N< groups in which alkyl is as defined above and acyl represents alkylcarbonyl; an example is the —(CH₂)₃—N(COCH₂)—(CH₂)₂NH(COCH₃) group;

carboxy-functional groups such as carboxyalkyl optionally interrupted with one or more oxygen or sulfur atoms, in which alkyl is as defined above; an example is the —CH₂—CH₂—S—CH₂—COOH group.

Examples of radicals R that may be mentioned include:

alkyl groups, preferably optionally halogenated C₁-C₁₀ alkyl, such as methyl, ethyl, octyl or trifluoropropyl;

aryl groups, preferably of C₆-C₁₃, such as phenyl;

amino-functional groups such as alkyl or aryl substituted with amino, alkyl preferably being of C₁-C₆ and aryl denoting a cyclic aromatic hydrocarbon-based group preferably of C₆-C₁₃ such as phenyl; examples of these are ethylamino and phenylamino;

amido-functional groups such as alkylcarbonylamino in which alkyl is preferably of C₁-C₆; an example of these is methylacetamido.

Concrete examples of “units D” that may be mentioned include: (CH₃)₂SiO; CH₃(CH═CH₂)SiO; CH₃(C₅H₅)SiO; (C₆H₅)₂SiO; CH₃(CH₂—CH₂—CH₂OH)SiO.

Concrete examples of “units M” that may be mentioned include. (CH₃)SiO_{1/2}; (CH₃)₂(OH)SiO_{1/2}: (CH₃)₂(CH═CH₂)SiO_{1/2}; [O—CH₃)═CH₂]₃SiO_{1/2}; [ON═C(CH₃)]₃SiO_{1/2}; (NH—CH₃)₃SiO_{1/2}; (NH—CO—CH₃)₃SiO_{1/2}.

Concrete examples of “units T” that may be mentioned include: CH₃SiO_{3/2}; (CH═CH₂)SiO_{3/2}.

When the silicones contain reactive and/or polar radicals R (such as OH, vinyl, allyl, hexenyl, aminoalkyl, etc.), these radicals generally represent not more than 5% of the weight of the silicone and preferably not more than 1% of the weight of the silicone.

Volatile oils, for instance hexamethyldisiloxane, octamethyldisiloxane, decamethyltetrasiloxane, dodecamethylpentasiloxane, tetradecamethylhexasiloxane, hexadecamethylhexasiloxane; heptamethyl-3[(trimethylsilyl)oxy}trisiloxane, hexamethyl-3,3-bis[(trimethylsilyl)oxy]trisiloxane; hexamethylcyclotrisiloxane, octamethylcyclotetrasiloxane, decamethylcyclopentasiloxane, dodecamethylcyclohexasiloxane, pentamethyl[(trimethylsilyl)oxy]cyclotrisiloxane, may preferably be used.

Similarly, nonvolatile silicones may be used, for instance polydimethylsiloxane and α,ω-bis(hydroxy)polydimethylsiloxane oils and gums and also polydimethylsiloxane, polyphenylmethylsiloxane and α,ω-bis(hydroxy)polydimethylsiloxane gums may be used.

α,ω-Bis(trimethyl)polydimethylsiloxane oils and α,ω-bis(hydroxy)polydimethylsiloxane oils are more particularly preferred.

As representative silicones that are most particularly suitable for the present invention, mention may be made especially of silicones of polydimethylsiloxane (dimethicone) and diphenyl dimethicone type.

The composition may especially comprise polymers other than the copolymer c) and, where appropriate, polyorganosiloxanes. They may be synthetic polymers or polymers of natural origin. They may especially be thickeners, surface-treating agents, especially conditioning agents, or agents that assist in the deposition of conditioning agents. Such polymers may especially be partially or totally water-soluble polymers.

As regards synthetic polymers, it may especially be:

an optionally crosslinked polyacrylate and/or methacrylate, optionally comprising hydrophobic units, where appropriate in the form of aqueous dispersions in which the polymer becomes dissolved by increasing the pH,

a cationic or potentially cationic synthetic polymer, or an amphoteric or ampholytic synthetic polymer. Such compounds are especially referenced under the “Polyquaternium” INCI names listed below.

Mention is made especially of:

crosslinked polyacrylates, for example polymers of Carbopol or Carbomer type sold by BF Goodrich or Noveon, Acritamer sold by Rita or Tego Carbomer sold by Goldschmidt, These compounds may be typically present in an amount of from 0 1% to 3% and preferably from 0.3% to 2% by weight relative to the composition. Mention may be made in particular of crosslinked copolymers of methacrylic acid and of a C₁-C₄ alkyl acrylate, such as the Carbopol Aqua-SF1 from Noveon;

the PEG-20 C₁₀-C₃₀ alkyl acrylate/aminoacrylate/itaconate copolymers sold by National Starch under the name Structure Plus. These compounds may typically be present in an amount of from 0.1% to 3% and preferably from 0.3% to 2% by weight relative to the composition;

viscosity modifiers, gelling agents or texturing agents, for instance anionic acrylic copolymers of Aculyn type sold by ISP or Rohm & Haas.

As commercial compounds that may be used, mention is made of

Carbopol ETD-2020, Noveon

Carbopol Aqua SF-1, Noveon

Carbopol 980, Noveon

Aculyn 22, Rohm & Haas

Structure Plus, National Starch.

As regards the polymers of natural origin, they may especially be cationic, nonionic or anionic derivatives, where appropriate hydrophobic. They may be, for example, polysaccharides or polysaccharide derivatives, keratin derivatives or proteins or protein derivatives. Cationic derivatives of natural polymers are especially referenced under the “Polyquaternium” INCI names listed below.

Mention may be made especially of polysaccharides and the noncationic derivatives thereof, such as cellulose derivatives, for instance hydroxypropylcellulose, carboxymethylcellulose, nonionic guar derivatives, for instance hydroxypropyl guar (for example the Jaguar HP products sold by Rhodia), locust been gum, tare gum or cassia gum, xanthan gum (for example the Rhodicare products sold by Rhodia), succinoglycans (for example Rheozan sold by Rhodia), alginates, carrageenans, chitin derivatives or any other polysaccharide with a texturing function. These polysaccharides and derivatives thereof may be incorporated alone or in synergistic combination with other polysaccharides. These compounds may typically be present in an amount of from 0.1% to 3% and preferably from 0.3% to 1% by weight relative to the composition.

Mention may be made in particular of

xanthan gum

succinoglycan

hydroxyethyl cellulose

hydroxypropyl guar

hydrolyzed keratins.

Additives are especially polymers of Polyquaternium type according to the INCI terminology familiar to those skilled in the art, for example chosen from the polymers of Table I below.

TABLE 1

| | Chemical nature and/or | Commercial |
|---|---|---|
| INCI name | CAS number | compounds |
| Polyquaternium-2 | CAS 63451-27-4 | Mirapol A15, |
| | | Rhodia |
| Polyquaternium-4 | CAS 92183-41-0 | Celquat L200, |
| | | H100, National |
| | | Starch |
| Polyquaternium-5 | CAS 26006-22-4 | |
| Polyquaternium-6 | DADMAC polymer | Merquat 1000, |
| | CAS 26062-79-3 | Nalco, |
| | | Mirapol 100, |
| | | Rhodia |
| Polyquaternium-7 | Copolymer of DADMAC | Merquat 5500, |
| | and of acrylamide | Nalco; Mirapol |
| | CAS 26590-05-6 | 550, Rhodia |
| Polyquaternium-10 | Hydroxyethylcellulose | Polymer JR 400, |
| | modified with | Amerchol; Celquat |
| | trimethylammoniums | SC230M or SC- |
| | | 240C, National |
| | | Starch |
| Polyquaternium-11 | Copolymers of | Gafquat 755N, |
| | vinylpyrrolidone and of | ISP; Luviquat |
| | quaternized | PQ11PN, BASF |
| | dimethylaminoethyl | |
| | methacrylate | |
| Polyquaternium-16 | CAS 29297-55-0 | Luviquat HM 552, |
| | | Luviquat FC 370, |
| | | BASF |
| Polyquaternium-17 | CAS 90624-75-2 | Mirapol AD1, |
| | | Rhodia |
| Polyquaternium-19 | CAS 110736-85-1 | |
| Polyquaternium-22 | Copolymer of DADMAC | Merquat 280, 281, |
| | and of acrylic acid | 298, Nalco |
| Polyquaternium-24 | Hydroxyethylcellulose | Quatrisoft LM200, |
| | modified with quaternary | Amerchol |
| | ammoniums containing | |
| | long alkyl chains | |
| Polyquaternium-27 | | Merquat 2001, |
| | | Nalco |
| Polyquaternium-28 | Copolymer of vinylpyr- | Gafquat HS 100, |
| | rolidone and of MAPTAC | BASF |
| Polyquaternium-29 | Chitosan derivative | Kytamer KCO, |
| | modified with propylene | Amerchol, |
| | oxide and quaternized with | Lexquat CH |
| | epichlorohydrin | |
| Polyquaternium-31 | CAS 136505-02-7 and | Hypan HQ |
| | 139767-67-7 | |
| Polyquaternium-32 | CAS 254429-19-7 | |
| Polyquaternium-37 | CAS 35429-19-7 | |
| Polyquaternium-39 | | Merquat 3300, |
| | | 3331, Nalco |
| Polyquaternium-44 | | Luviquat Care, |
| | | BASF |
| Polyquaternium-46 | copolymers of vinyl- | Luviquat Hold, |
| | caprolactam, vinylpyr- | BASF |
| | rolidone, and cationized | |
| | vinylimidazole | |
| Guar hydroxypropyl- | | Jaguar C13S, |
| ammonium chloride | | C14S, C17, Excel, |
| | | Rhodia |
| Hydroxypropyl | | Jaguar C162, |
| guar hydroxy- | | Rhodia |
| propylammonium | | |
| chloride | | |
| Polyquaternium-67 | Hydroxyethylcellulose | Softcat SL, |
| | modified with quaternary | Amerchol |
| | ammoniums containing | |
| | long alkyl chains and with | |
| | short-chain quaternary | |
| | ammoniums | |
| Polymethacryl- | MAPTAC polymer | Polycare 133, |
| amidopropyl- | | Rhodia |
| trimonium chloride | | |
| Acrylamidopropyl- | | Salcare SC-60, |
| trimonium chloride/ | | Ciba |
| acrylamide copolymer | | |

As nacreous agents and/or agents possibly forming insoluble solids forming a network in the composition, they may especially be mono- and/or diesters of fatty acids of ethylene glycol, the fatty acids preferably being of C₁₆-C₁₈. In particular, it may be ethylene glycol distearate (EGDS), for example sold by Rhodia as a concentrate with other ingredients under the name Mirasheen. This compound may be typically present in an amount of from 3% to 10% and preferably from 5% to 8% by weight relative to the composition. Such a compound may be introduced into the composition via any known method, especially by cold mixing, where appropriate in crystalline form, or by hot mixing, where appropriate with subsequent crystallization. It may be introduced in the form of a mixture with other compounds, especially surfactants. Mention is made especially of distearyl ether, ethylene glycol distearate (EGDS) (INCI: glycol distearate), polyethoxylated and/or polypropoxylated stearates or distearates, for example PEG-3 distearates, PEG/PPG distearates, PEG-200 distearates and PEG-100 stearates. Commercial products that may be used are especially Mirasheen CP 820, Rhodia; Euperlan PK-3000 AM, Cognis; Euperlan PK-771 BENZ, Cognis: Genapol TS, Clariant (INCI PEG-3 distearate).

Preserving agents such as methyl, ethyl, propyl and butyl esters of p-hydroxybenzoic acid, or sodium benzoate, may also be introduced into the aqueous cosmetic compositions according to the invention, generally in a proportion of from 0.01% to 3% by weight. Mention is made especially of the products sold under the name Germaben®, or Kathon®, or any chemical agent that prevents the proliferation of bacteria or molds and that is conventionally used in cosmetic compositions.

The composition according to the invention may be a hair dye composition. Such compositions are known to those skilled in the art. It is pointed out that hair dye compositions may be formed from several hair dyeing products, intended to be mixed together by the user. In the present patent application, unless otherwise mentioned or particularly specified, the term “hair dye composition” covers both a whole composition, or a product intended to be mixed with another by the user. In the present patent application, the term “hair dyeing” covers any modification of the color of the hair, whether it is actual dyeing, bleaching, or a combination of bleaching and dyeing.

The hair dye composition may comprise an oxidation base (oxidation dye precursors). It may comprise an oxidizing agent. It may comprise a coupler (coloration modifier). It may comprise a direct dye. The composition comprises a cosmetically acceptable vector. The composition may also comprise adjuvants.

According to one embodiment, it is a composition for long-lasting dyeing comprising an oxidation base, an oxidizing agent, and optionally a coupler, preferably in the form of two products to be combined, one product comprising the oxidation base and one product comprising the oxidizing agent.

According to one embodiment, it is a composition for temporary or long-lasting dyeing comprising a direct dye, and optionally an oxidizing agent.

According to one embodiment, it is a composition for bleaching or lightening the hair, comprising an oxidizing agent.

As direct dyes, mention may be made of neutral acidic or cationic nitrobenzene dyes, neutral, acidic or cationic azo direct dyes, neutral, acidic or cationic quinone and in particular anthraquinone direct dyes, azine direct dyes, methine direct dyes, tetraazapentamethine direct dyes, triarylmethane direct dyes, indoamine direct dyes and natural direct dyes.

As oxidizing agents, mention may be made of hydrogen peroxide, urea peroxide, alkali metal bromates, persalts such as perborates and persulfates, peracids, and enzymes, especially peroxidases, 2-electron oxidoreductases, and 4-electron oxygenases.

As couplers, mention may be made of meta-phenylenediamines, meta-aminophenols, meta-diphenols, naphthalene-based couplers and heterocyclic couplers.

Other details or advantages of the invention will emerge in the light of he examples that follow, without any limiting nature.

### EXAMPLES

In the examples, the formulations comprise organized surfactants at least partly in the form of giant micelles.

### Example 1

### Preparation of a 99/1 mol/mol Poly(SPP/Lauryl Methacrylate) Copolymer

The synthesis proceeds in two steps: preparation of an aqueous mixture comprising the monomers, followed by copolymerization.

Preparation of an Aqueous Mixture Comprising the Monomers:

1.51 g of lauryl methacrylate, 54.5 g of a 30% solution of sodium dodecylsulfate (“SDS”), 470 g of water and 9.94 g of sodium sulfate are added to a 1 liter glass beaker with magnetic stirring. Stirring is continued until a clear micelle solution is obtained (Mixture 1—aqueous fluid B), 172.2 g of SPP and 172.2 g of water are added to a 500 ml glass beaker with magnetic stirring. Stirring is continued until a clear solution is obtained (Mixture 2—aqueous solution A). Mixture 2 is then introduced into mixture 1 with magnetic stirring. Stirring is continued until a clear micelle solution is obtained (Mixture 3). All these mixing steps are performed at room temperature.

Copolymerization:

100 g of water are added to a 1.5 liter jacketed glass reactor equipped with a mechanical stirrer, a condenser and temperature regulation by means of a heating bath. The temperature of the reaction medium is brought to 80° C. while flushing with nitrogen. At 80° C., addition is then simultaneously performed of mixture 3 over 3 hours and a solution of 0.81 g of 2,2′-azobis(2-methylpropionamidine)dihydrochloride in 10 g of water over 4 hours 15 minutes. At the end of these additions, a solution of 0.48 g of 2,2′-azobis(2-methylpropionamidine)dihydrochloride in 10 g of water is added over 3 hours. The reaction medium is then cooled to room temperature.

### Example 2

### Preparation of a 97/3 mol/mol Poly(SPP/Lauryl Methacrylate) Copolymer

The synthesis proceeds in two steps: preparation of an aqueous mixture comprising the monomers, followed by copolymerization.

Preparation of an Aqueous Mixture Comprising the Monomers:

4.34 g of lauryl methacrylate, 81.8 g of a 30% solution of sodium dodecylsulfate (“SDS”), 461.6 g of water and 9.94 g of sodium sulfate are added to a 1 liter glass beaker with magnetic stirring. Stirring is continued until a clear micelle solution is obtained (Mixture 1—aqueous fluid B). 161.2 g of SPP and 161.2 g of water are added to a 500 ml glass beaker with magnetic stirring. Stirring is continued until a clear solution is obtained (Mixture 2—aqueous solution A). Mixture 2 is then introduced into mixture 1 with magnetic stirring. Stirring is continued until a clear micelle solution is obtained (Mixture 3). All these mixing steps are performed at room temperature.

Copolymerization:

100 g of water are added to a 1.5 liter jacketed glass reactor equipped with a mechanical stirrer, a condenser and temperature regulation by means of a heating bath. The temperature of the reaction medium is brought to 80° C. while flushing with nitrogen. At 80′ C., addition is then simultaneously performed of mixture 3 over 3 hours and a solution of 0.77 g of 2,2′-azobis(2-methylpropionamidine) dihydrochloride in 10 g of water over 4 hours 15 minutes. At the end of these additions, a solution of 0.46 g of 2,2′-azobis(2-methylpropionamidine)dihydrochloride in 10 g of water is added over 3 hours. The reaction medium is then cooled to room temperature.

### Example 3

### Preparation of a 99/1 mol/mol Poly(SPP/Purified Plex6877-O) Copolymer

The synthesis proceeds in two steps: preparation of an aqueous mixture comprising the monomers, followed by copolymerization.

Preparation of an Aqueous Mixture Comprising the Monomers:

9.33 g of purified Plex6877-O, 4.09 g of a 30% solution of sodium dodecylsulfate (“SDS”) and 487.7 g of water are added to a 1 liter glass beaker with magnetic stirring. Stirring is continued until a clear micelle solution is obtained (Mixture 1—aqueous fluid B). 189.4 g of SPP and 189.4 g of water are added to a 500 ml glass beaker with magnetic stirring. Stirring is continued until a clear solution is obtained (Mixture 2—aqueous solution A). Mixture 2 is then introduced into mixture 1 with magnetic stirring. Stirring is continued until a clear micelle solution is obtained (Mixture 3). All these mixing steps are performed at room temperature.

Copolymerization:

100 g of water are added to a 1.5 liter jacketed glass reactor equipped with a mechanical stirrer, a condenser and temperature regulation by means of a heating bath. The temperature of the reaction medium is brought to 80° C. while flushing with nitrogen. At 80° C., addition is then simultaneously performed of mixture 3 over 3 hours and a solution of 0.89 g of 2.2′-azobis(2-methylpropionamidine)dihydrochloride in 10 g of water over 4 hours 15 minutes. At the end of these additions, a solution of 0.53 g of 2,2′-azobis(2-methylpropionamidine)dihydrochloride in 10 g of water is added over 3 hours. The reaction mixture is then cooled to room temperature.

### Example 4

### Preparation of a 29.5/67.513 mol/mol Poly(SPP/Acrylamide/Lauryl Methacrylate) Copolymer

The synthesis proceeds in two steps: preparation of an aqueous mixture comprising the monomers, followed by copolymerization.

Preparation of an Aqueous Mixture Comprising the Monomers:

3.93 g of lauryl methacrylate, 73.1 g of a 30% solution of sodium dodecylsulfate (“SDS”). 5.4 g of sodium sulfate and 220.1 g of water are added to a 1 liter glass beaker with magnetic stirring. Stirring is continued until a clear micelle solution is obtained (Mixture 1—aqueous fluid B). 57 g of SPP, 57 g of water and 63.5 g of 50% acrylamide are added to a 500 ml glass beaker with magnetic stirring. Stirring is continued until a clear solution is obtained (Mixture 2—aqueous solution A). Mixture 2 is then introduced into mixture 1 with magnetic stirring. Stirring is continued until a clear micelle solution is obtained (Mixture 3). All these mixing steps are performed at room temperature.

Copolymerization:

100 g of water are added to a 1 liter jacketed glass reactor equipped with a mechanical stirrer, a condenser and temperature regulation by means of a heating bath. The temperature of the reaction medium is brought to 80° C. while flushing with nitrogen. At 80° C., addition is then simultaneously performed of mixture 3 over 3 hours and a solution of 0.9 g of 2,2′-azobis(2-methylpropionamidine)dihydrochloride in 10 g of water over 4 hours 15 minutes. At the end of these additions, a solution of 0.54 g of 2,2′-azobis(2-methylpropionamidine)dihydrochloride in 10 g of water is added over 3 hours. The reaction mixture is then cooled to room temperature.

Total volume=0.544 L

n lauryl methacrylate=0.015 mol

n SDS=0.076 mol

n SOS−cmc SDS=0.076−0.007×0.544=0.072 mol

nH=12.9

### Example 5

### Formulations and Viscosity Measurements—Iso-Concentration of Salt and Polymer—Modulation of the Composition of the Polymer

Formulations are prepared comprising the copolymers, along with comparative formulations without copolymer.

The products obtained from the syntheses described in Examples 1 to 4 are placed in watch glasses and then placed in an oven at 105° C. overnight so as to free them of their water. The dry residues are recovered in the form of brittle films, which are then reduced to fine powder in a mortar so as to be easily manipulable.

Formulation A (Comparative)

The following reference formulation, representative of standard shower gel or shampoo formulations, and not containing any polymer, is prepared.

The process is performed in a 200 ml glass beaker, by addition with moderate stirring (150 rpm) of:
36.00 g of deionized water
0.06 g of Kathon CG (preserving agent containing 100% by weight of active material): after dissolution of the Kathon CG
61.65 g of Empicol ESB/3M, solution in water at 27.3% by weight of sodium lauryl ether sulfate active material
7.97 g of Mirataine BET C30, solution in water at 30.1% by weight of cocamidopropylbetaine active material; after homogenization of the solution,
1.92 g of sodium chloride (NaCl) salt predissolved in 12.40 g of deionized water.

In this polymer-free formulation, the anionic surfactant Empicol ESB/3M represents 14% by weight, the amphoteric surfactant Mirataine BET 030 represents 2% by weight, and the salt NaCl represents 1.6% by weight.

Formulations B, C, D and E Comprising the Products Obtained from Examples 1, 2, 3 and 4 (Respectively)

The following formulations are prepared, containing one of the 4 polymers of Examples 1 to 4. These are formulations identical to formulation A in addition containing the polymer. The process is performed in a 200 ml glass beaker, by addition with moderate stirring (150 rpm) of:
36.00 g of deionized water
0.06 g of Kathon CG (preserving agent containing 100% by weight of active material); after dissolution of the Kathon CG,
61.65 g of Empicol ESB/3M, solution in water at 27.3% by weight of sodium lauryl ether sulfate active material
7.97 g of Mirataine BET C30, solution in water at 30.1% by weight of cocamidopropylbetaine active material;
after homogenization of the solution addition of:
0.36 g of dry polymer obtained from Example 1, 2, 3 or 4;
after total dissolution of the polymer, addition with stirring of
1.92 g of sodium chloride (NaCl) salt predissolved in 12.04 g of deionized water.

In these formulations, the anionic surfactant Empicol ESB/3M represents 14% by weight, the amphoteric surfactant Mirataine BET C30 represents 2% by weight, the salt NaCl represents 1.6% by weight and the added polymer represents 0.3% by weight.

The Brookfield viscosities of the polymer-free reference formulation (formulation A), and of formulations B, C, D and E containing 0.3% by weight of one of the four polymers of Examples 1 to 4 are compared. The viscosity is measured with a Brookfield viscometer, at 10 rpm (revolutions per minute) with an RV3, 4, 5 or 6 measuring needle (spindle), adapted according to the viscosity of the formulation to be characterized. The viscosity measurement is given in centipoises (cP).

The viscosity at 10 rpm RV5 spindle of the polymer-free comparative formulation A is 2000 cP.

The viscosity at 10 rpm RV5 spindle of formulation B containing 0.3% by weight of the polymer of Example 1 is 2880 cP,

The viscosity at 10 rpm RV5 spindle of formulation C containing 0.3% by weight of the polymer of Example 2 is 4300 cP,

The viscosity at 10 rpm RV5 spindle of formulation D containing 0.3% by weight of the polymer of Example 3 is 6860 cP,

The viscosity at 10 rpm RV5 spindle of formulation E containing 03% by weight of the polymer of Example 4 is 15700 cP.

The various copolymers allow the viscosity to be increased. It is possible to modulate the viscosity by modulating the copolymer composition.

### Example 6

### Formulations and Viscosity Measurements—Reduction of the Amount of Salt

The following two polymer-free comparative formulations are prepared. The process is performed in a 200 ml glass beaker, by addition with moderate stirring (150 rpm).

Formulation F (Comparative)
36.00 g of deionized water
0.06 g of Kathon CG (preserving agent at 100% by weight of active material);
after dissolution of the Kathon CG, addition of
30.83 g of Empicol ESB/3M, solution in water at 27.3% by weight of sodium lauryl ether sulfate active material
3.98 g of Mirataine BET C30, solution in water at 30.1% by weight of cocamidopropylbetaine active material;
after homogenization of the solution, addition of
3.60 g of sodium chloride (NaCl) salt predissolved in 45.53 g of deionized water.

In this polymer-free formulation, the anionic surfactant Empicol ESB/3M represents 7% by weight, the amphoteric surfactant Mirataine BET C30 represents 1% by weight and the salt NaCl represents 3.0% by weight.

Formulation G (Comparative)
36.00 g of deionized water
0.06 g of Kathon CG (preserving agent at 100% by weight of active material);
after dissolution of the Kathon CG, addition of
30.83 g of Empicol ESB/3M, solution in water at 27.3% by weight of sodium lauryl ether sulfate active material
3.98 g of Mirataine BET C30, solution in water at 30.1% by weight of cocamidopropylbetaine active material;
after homogenization of the solution, addition of
4.80 g of sodium chloride (NaCl) salt predissolved in 44.33 g of deionized water.

In this polymer-free formulation, the anionic surfactant Empicol ESB/3M represents 7% by weight, the amphoteric surfactant Mirataine BET C30 represents 1% by weight and the salt NaCl represents 4.0% by weight.

Formulation H (Identical to Formulation F with, in Addition, the Polymer)
36.00 g of deionized water
0.06 g of Kathon CG (preserving agent at 100% by weight of active material);
after dissolution of the Kathon CG, addition of
30.83 g of Empicol ESB/3M, solution in water at 27.3% by weight of sodium lauryl ether sulfate active material
3.98 g of Mirataine BET C30, solution in water at 30.1% by weight of cocamidopropylbetaine active material;
after homogenization of the solution, addition of
0.36 g of dry polymer obtained from Example 4; after total dissolution of the polymer, addition with stirring of
3.60 g of sodium chloride (NaCl) salt predissolved in 45.17 g of deionized water.

In this formulation, the anionic surfactant Empicol ESB/3M represents 7% by weight, the amphoteric surfactant Mirataine BET C30 represents 1% by weight and the salt NaCl represents 3.0% by weight and the polymer represents 0.3% by weight.

The Brookfield viscosities are compared, at 10 rpm (revolutions per minute) with an RV5 measuring needle (spindle).

The viscosity at 10 rpm RV5 spindle of the polymer-free comparative formulation F containing 3% by weight of NaCl is 620 cP.

The viscosity at 10 rpm RV5 spindle of the polymer-free comparative formulation G containing 4% by weight of NaCl is 8000 cP.

The viscosity at 10 rpm RV5 spindle of formulation H containing 0.3 by weight of the polymer of Example 4 and containing 3% by weight of NaCl is 8000 cP.

The copolymer of the invention makes it possible to reduce the salt content and to compensate for the loss of viscosity. Less polymer is added than surfactant is subtracted.

### Example 7

### Preparation of a 29.5/67.513 mol/mol Poly(SPP/Acrylamide/Lauryl Methacrylate Copolymer—nH=2

The process is performed as described in Example 4, by increasing the SDS amount to 469 g of a 30% solution, so as to obtain a number nH=2.

### Example 8

### Preparation of a 29.5/67.5/3 mol/mol Poly(SPP/Acrylamide/Lauryl Methacrylate) Copolymer—nH=6.4

The process is performed as described in Example 4, by increasing the amount of SDS to 146.2 g of a 30% solution, so as to obtain a number nH=6.4.

### Example 9

### Preparation of a 29.5/67.5/3 mol/mol Poly(SPP/Acrylamide/Lauryl Methacrylate) Copolymer—nH=10.9

The process is performed as described in Example 4, by increasing the amount of SDS to 87.7 g of a 30% solution, so as to obtain a number nH=10.9.

### Example 10

### Formulations and Viscosity Measurements—Modulation of the Number nH

The products obtained from the syntheses described in Examples 7 to 9 are used as obtained.

Their real dry extract is measured at 27.94%, 22.03% and 22.57% by weight for the products obtained from syntheses 7, 8 and 9, respectively.

Formulation I (Based on the Product Obtained from the Synthesis of Example 7)

The following formulation based on the product obtained from the synthesis of Example 7 is prepared. The process is performed in a 200 ml glass beaker, by addition with moderate stirring (150 rpm) of:
36.00 g of deionized water
0.06 g of Kathon CG (preserving agent at 100% by weight of active material);
after dissolution of the Kathon CG, addition of
61.65 g of Empicol ESB/3M, solution in water at 27.3% by weight of sodium lauryl ether sulfate active material;
7.97 g of Mirataine BET C30, solution in water at 30.1% by weight of cocamidopropylbetaine active material;
after homogenization of the solution, addition of
2.15 g of polymer obtained from Example 7 at 27.94% by weight of ctive material;
after total dissolution of the polymer, addition with stirring of
1.92 g of sodium chloride (NaCl) salt predissolved in 10.25 g of deionized water.

In these formulations, the anionic surfactant Empicol ESB/3M represents 14% by weight, the amphoteric surfactant Mirataine BET C30 represents 2% by weight, the salt NaCl represents 1.6% by weight and the added polymer represents 0.5% by weight.

Formulation J (Based on the Product Obtained from the Synthesis of Example 8)

The following formulation based on the product obtained from the synthesis of Example 7 is prepared. The process is performed in a 200 ml glass beaker, by addition with moderate stirring (150 rpm) of:
36.00 g of deionized water
0.06 g of Kathon CG (preserving agent at 100% by weight of active material);
after dissolution of the Kathon CG, addition of
61.65 g of Empicol ESB/3M, solution in water at 27.3% by weight of sodium lauryl ether sulfate active material;
7.97 g of Mirataine BET C30, solution in water at 30.1% by weight of cocamidopropylbetaine active material;
after homogenization of the solution, addition of
2.72 g of polymer obtained from Example 8 at 22.03% by weight of active material;
after total dissolution of the polymer, addition with stirring of
1.92 g of sodium chloride (NaCl) salt predissolved in 9.67 g of deionized water.

In these formulations, the anionic surfactant Empicol ESB/3M represents 14% by weight. the amphoteric surfactant Mirataine BET C30 represents 2% by weight, the salt NaCl represents 1.6% by weight and the added polymer represents 0.5% by weight.

Formulation K (Based on the Product Obtained from the Synthesis of Example 9)

The following formulation based on the product obtained from the synthesis of Example 7 is prepared. The process is performed in a 200 ml glass beaker, by addition with moderate stirring (150 rpm) of:
36.00 g of deionized water
0.06 g of Kathon CG (preserving agent at 100% by weight of active material);
after dissolution of the Kathon CG, addition of
61.65 g of Empicol ESB/3M, solution in water at 27.3% by weight of sodium lauryl ether sulfate active material;
7.97 g of Mirataine BET C30, solution in water at 30.1% by weight of cocamidopropylbetaine active material;
after homogenization of the solution, addition of
2.66 g of polymer obtained from Example 9 at 22.57% by weight of active material;
after total dissolution of the polymer, addition with stifling of
1.92 g of sodium chloride (NaCl) salt predissolved in 974 g of deionized water.

In these formulations, the anionic surfactant Empicol ESB13M represents 14% by weight, the amphoteric surfactant Mirataine BET C30 represents 2% by weight, the salt NaCl represents 1.6% by weight and the added polymer represents 0.5% by weight.

The Brookfield viscosities are compared, at 10 rpm (revolutions per minute) with an RV5 measuring needle (spindle).

It is recalled that the viscosity at 10 rpm RV5 spindle of the polymer-free reference formulation A is 2000 cP.

The viscosity at 10 rpm RV5 spindle of formulation I containing 0.5% by weight of the polymer of Example 7 is 8080 cP.

The viscosity at 10 rpm RV5 spindle of formulation J containing 0.5% by weight of the polymer of Example 8 is 23 920 cP

The viscosity at 10 rpm RV5 spindle of formulation K containing 0.5% by weight of the polymer of Example 9 is 15 960 cP.

The various copolymers allow the viscosity to be increased. It is possible to modulate the effect on the viscosity by modulating the number nH. A number nH of 6.4 affords greater effects than numbers nH of 2 or 10.9.

### Example 11

### Formulations and Viscosity Measurements—Reduction of the Surfactant Content

The following two stock formulations are prepared.

Formulation L (Stock Formulation)

The process is performed in a 200 ml glass beaker, by addition with moderate stirring (150 rpm) of:
120.34 g of deionized water
0.06 g of Kathon CG (preserving agent at 100% by weight of active material); after dissolution of the Kathon CG, addition of
306.5 g of Rhodapex ESB-3A2, solution in water at 25.7% by weight of sodium lauryl ether sulfate active material;
36.6 g of Mirataine BET C30, solution in water at 30.77% by weight of cocamidopropylbetaine active material;
after homogenization of the solution, addition of
36.5 g of polymer obtained from Example 8 at 22.03% by weight of active material, with stirring until fully dissolved.

In this formulation, the anionic surfactant Rhodapex ESB-3A2 represents 15.75% by weight, the amphoteric surfactant Mirataine BET C30 represents 2.25% by weight and the polymer represents 1.61% by weight.

Formulation M (Stock Formulation)

The process is performed in a 200 ml glass beaker, by addition with moderate stirring (150 rpm) of:
156.84 g of deionized water
0.06 g of Kathon CG (preserving agent at 100% by weight of active material);
after dissolution of the Kathon CG, addition of
306.5 g of Rhodapex ESB-3A2, solution in water at 25.7% by weight of sodium lauryl ether sulfate active material;
36.6 g of Mirataine BET C30, solution in water at 30.77% by weight of cocamidopropylbetaine active material.

In this formulation, the anionic surfactant Rhodapex ESB-3A2 represents 15.7 by weight and the amphoteric surfactant Mirataine BET C30 represents 2.25% by weight.

The following formulations are prepared by mixing formulations L and M. The process is performed in a 200 ml glass beaker, by addition with moderate stirring (150 rpm).

Formulation N

The following are introduced into a 200 ml glass beaker with moderate stirring (150 rpm):
18.67 g of formulation L
88 g of formulation M;
after homogenization for a few minutes, addition of
1.92 g of sodium chloride (NaCl) salt predissolved in 11.41 g of deionized water.

In this formulation, the anionic surfactant Rhodapex ESB-3A2 represents 14% by weight, the amphoteric surfactant Mirataine BET C30 represents 2% by weight for a total surfactant content of 16% by mass. The salt NaCl represents 1.6% by weight and the polymer represents 0.25% by weight.

Formulation O

The following are introduced into a 200 ml glass beaker with moderate stirring (150 rpm):
37.33.67 g of formulation L
53.33 g of formulation M;
after homogenization for a few minutes, addition of
1.92 g of sodium chloride (NaCl) salt predissolved in 27.41 g of deionized water.

In this formulation, the anionic surfactant Rhodapex ESB-3A2 represents 11.9% by weight. the amphoteric surfactant Mirataine BET C30 represents 1.7% by weight for a total surfactant content of 13.6% by weight, resulting in a 15% reduction in surfactant relative to formulation O. The salt NaCl represents 1.6% by weight and the polymer represents 0.50% by weight.

Formulation P

The following are introduced into a 200 ml glass beaker with moderate stirring (150 rpm):
56 g of formulation L
18.67 g of formulation M;
after homogenization for a few minutes, addition of
1.92 g of sodium chloride (NaCl) salt predissolved in 43.41 g of deionized water.

In this formulation, the anionic surfactant Rhodapex ESB-3A2 represents 9.8% by weight, the amphoteric surfactant Mirataine BET C30 represents 1.4% by weight for a total surfactant content of 11.2% by weight, resulting in a 30% reduction in surfactant relative to formulation O. The salt NaCl represents 1.6% by weight and the polymer represents 0.75% by weight.

The Brookfield viscosities are compared, at 10 rpm (revolutions per minute) with an RV4 measuring needle (spindle).

It is recalled that the viscosity at 10 rpm RV5 spindle of the polymer-free reference formulation A is 2000 cP.

The viscosity at 10 rpm RV5 spindle of formulation N containing in total 16.0% by weight of surfactants and 0.25% by weight of the polymer of Example 8 is 7480 cP.

The viscosity at 10 rpm RV5 spindle of formulation 0 containing in total 13.6% by weight of surfactants and 0.50% by weight of the polymer of Example 8 is 8380 cP.

The viscosity at 10 rpm RV5 spindle of formulation P containing in total 11.2% by weight of surfactants and 0.75% by weight of the polymer of Example 8 is 8440 cP.

The copolymer of the invention makes it possible to reduce the surfactant content and at the same time to increase the viscosity. Less polymer is added than surfactant is subtracted.

### Example 12

### Formulations Comprising a Structuring Agent and Viscosity Measurements

The following stock formulation is prepared.

Formulation Q (Stock Formulation)

The following are introduced with slow stirring (50 rpm) into a glass beaker heated on a water bath at 70° C.:
81.4 g of ammonium lauryl ether Inter EAZ70 at 70% by weight of active material
7.3 g of cocomonoethanolamide Alkamide MEA, a solid at 100% active material;
after homogenization, addition of
363 g of deionized water; after obtaining a transparent homogeneous liquid,
48.4 g of cocamidopropylbetaine Mirataine BET-C30 at 30.77% by weight of active material.

The pH is equilibrated to about 6.5 with a few drops of a concentrated citric acid solution. In this formulation, the anionic surfactant Inter EAZ70 represents 11.4% by weight, the amphoteric surfactant Mirataine BET C30 represents 3.0% by weight and the neutral surfactant Alkamide MEA represents 1.45% by weight, for a total surfactant content of 15.8% by weight.

The following polymer-free reference formulation is prepared.

Formulation R (Comparative)

The following are introduced with slow stirring (50 rpm) into a glass beaker heated on a water bath at 70° C.:
99.2 g of formulation Q
0.06 g of Kathon CG (preserving agent at 100% by weight of active material);
after dissolution of the Kathon CG, addition of
20.7 g of deionized water.

In this salt-free and polymer-free reference formulation, the anionic surfactant Inter EAZ70 represents 9.4% by weight, the amphoteric surfactant Mirataine BET C30 represents 2.5% by weight and the neutral surfactant Alkamide MEA represents 1.2% by weight, for a total surfactant content of 13.1% by weight.

The following polymer-free reference formulation containing salt is prepared.

Formulation S (Comparative)

The following are introduced with slow stirring (50 rpm) into a glass beaker heated on a water bath at 70° C.:
99.2 g of formulation Q
0.06 g of Kathon CG (preserving agent at 100% by weight of active material);
after dissolution of the Kathon CG, addition of
2.72 g of ammonium chloride NH₄Cl salt at 100% active material dissolved in 19.2 g of deionized water.

In this polymer-free reference formulation with salt, the anionic surfactant Inter EAZ70 represents 9.4% by weight, the amphoteric surfactant Mirataine BET C30 represents 2.5% by weight and the neutral surfactant Alkamide MEA represents 1.2% by weight, for a total surfactant content of 13.1% by weight. The salt NH₄Cl represents 1.25% by weight.

The following salt-free reference formulation containing polymer is prepared.

Formulation T

The following are introduced with slow stirring (50 rpm) into a glass beaker heated on a water bath at 70° C.:
99.2 g of formulation
0.06 g of Kathon CG (preserving agent at 100% by weight of active material);
after dissolution of the Kathon CG,
18.0 g of deionized water
2.72 g of polymer obtained from Example 8 at 22.03% by weight of active material, with stirring until dissolution is complete.

In this formulation, the anionic surfactant Inter EAZ70 represents 9.4% by weight, the amphoteric surfactant Mirataine BET C30 represents 2.5% by weight and the neutral surfactant Alkamide MEA represents 1.2% by weight, for a total surfactant content of 13.1% by weight. The polymer represents 0.5% by weight.

The Brookfield viscosities are compared, at 10 rpm (revolutions per minute) with an RV3 or 6 measuring needle (spindle), adapted according to the viscosity level of the formulation.

The viscosity at 10 rpm RV3 spindle of the reference formulation R not containing any salt or polymer is 1660 cP.

The viscosity at 10 rpm RV6 spindle of the reference formulation S containing 1.25% by weight of NH₄Cl but no polymer is 31 700 cP.

The viscosity at 10 rpm RV6 spindle of formulation T containing 0.5% by weight of polymer obtained from Example 8 but no salt is 67 400 cP.

1-25. (canceled)

26. An aqueous cosmetic composition comprising:
a) a surfactant system,
b) optionally at least one salt, and
c) at least one copolymer comprising:
units A_{Z} comprising a betaine group comprising a cationic group and an anionic group other than a carboxylate group,
units B comprising hydrophobic units B_{phobic} and/or amphiphilic units Bₐₘₚₕᵢ, and
optionally other hydrophilic units Aₒₜₕₑᵣ.

27. The composition of claim 26, wherein the composition comprises organized phases of surfactant(s).

28. The composition of claim 26, wherein the surfactant system comprises an anionic surfactant and/or a nonionic surfactant.

29. The composition of claim 27, wherein said organized phases of surfactant(s) comprise giant micelles.

30. The composition of claim 26, wherein:
the composition optionally further comprises a structuring agent, and
the weight ratio of the amount of copolymer c) to the, amount of surfactant(s) and optional structuring agent is less than 0.0625.

31. The composition of claim 26, wherein copolymer c) comprises a macromolecular chain. A comprising units A_{Z}.

32. The composition of claim 31, wherein, the number of hydrophobic units n_{H} in the group B_{phobic} is less than 2.6.

33. The composition of claim 31, wherein the number of hydrophobic units n_{H} in the group B_{phobic} is greater than or equal to 2.6.

34. The composition of claim 26, wherein copolymer c) comprises a copolymer obtainable by controlled micelle polymerization.

35. The composition of claim 26, wherein the mole ratio of the sum of the units A_{Z} and Aₒₜₕₑᵣ to the units B ranges from 1/99 to 99.9/0.1.

36. The composition of claim 35, wherein the mole ratio ranges from 80/20 to 99.5/0.5.

37. The composition of claim 36, wherein the mole ratio ranges from 95/5 to 99.5/0.5.

38. The composition of claim 26, wherein:
the copolymer comprises units A_{Z} and units Aₒₜₕₑᵣ, and
the mole ratio of units A_{Z} to the total of units Aₒₜₕₑᵣ ranges from 99/1 to 1/99.

39. The composition of claim 26, wherein the units A_{Z} are derivable from at least one betaine monomer comprising:
dialkylammonium alkyl acrylate, methacrylate alkyl sulfonate, phosphonate acrylamide, or phosphonate methacrylamide;
a heterocyclic betaine monomer;
a dialkylammonium alkyl allyl alkylsulfonate or phosphonate;
a dialkylammonium alkyl, styrene alkylsulfonate or phosphonates;
a betaine derivable from a diene or ethylenically unsaturated anhydride;
a betaine derivable from a cyclic acetal; or
a phosphobetaine of formula:

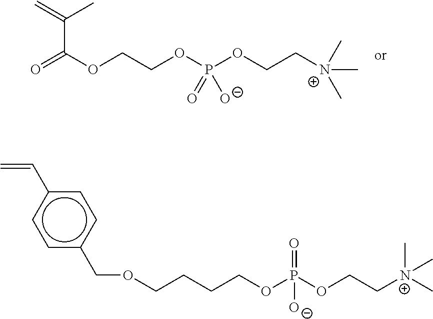

40. The composition of claim 39, wherein the units A_{Z} are derivable from at least one betaine monomer comprising:
sulfopropyldimethylammoniumethyl methacrylate
sulfoethyldimethylammoniumethyl methacrylate
sulfobutyldimethylammoniumethyl methacrylate
sulfohydroxypropyldimethylammoniumethyl methacrylate
sulfopropyldimethylammoniumpropylacrylamide
sulfopropyldimethylarnmoniumpropylmethacrylamide
sulfopropyldiethylammoniumethyl methacrylate
sulfohydroxypropyldimethylammoniumpropylmethacrylarnide, or
sulfohydroxypropyldiethylammoniumethyl methacrylate.

41. The composition of claim 39, wherein the heterocyclic betaine monomer comprises:
a sulfobetaine derivable from piperazine,
a sulfobetaine derivable from 2-vinylpyridine or 4-vinylpyridine, or
a 1-vinyl-3-(3-sulfopropyl)imidazolium betaine.

42. The composition of claim 26, wherein the units A_{Z} are derivable from at least one betaine monomer derivable from a chemical modification of units of a precursor polymer.

43. The composition of claim 42, wherein, said chemical modification comprises modification of a polymer comprising pendent amine functions with a sulfonated electrophilic compound.

44. The composition of claim 26, wherein the units A_{Z} comprise:

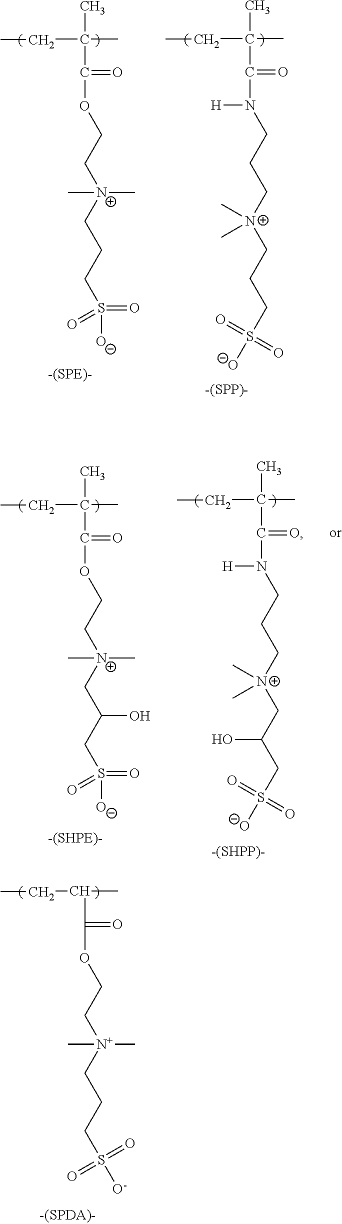

45. The composition of claim 26, wherein the copolymer comprises units Aₒₜₕₑᵣ and the units Aₒₜₕₑᵣ are derivable from hydrophilic monomers comprising:
neutral units A_{N}, derivable from neutral monomers,
cationic or potentially cationic units A_{C}, derivable from cationic or potentially cationic monomers,
anionic or potentially anionic units A_{A}, derivable from anionic or potentially anionic monomers, or
a mixture or combination thereof.

46. The composition of claim 45, wherein, the units A_{N} comprise units derivable from a monomer comprising:
a hydroxyalkyl ester of an α,β-ethylenically unsaturated acid,
an α,β-ethylenically unsaturated amide,
an α,β-ethylenically unsaturated, monomer bearing a water-soluble polyoxyalkylene segment,
a vinyl alcohol
a vinyllactam,
an α,β-ethylenically unsaturated ureido monomer, or
a mixture or combination thereof.

47. The composition of claim 46, wherein the units A_{N} comprise units derivable from a monomer comprising:
a hydroxyethyl acrylate, hydroxypropyl acrylate, hydroxyethyl methacrylate, hydroxypropyl methacrylate, or glycerol monomethacrylate;
an acrylamide, methacrylamide, N-methylolacrylamide, dimethylacylamide, or dimethylmethacrylamide;
a vinylpyrrolidone;
a 2-imidazolidinoneethylmethacrylamide; or
a mixture or combination thereof.

48. The composition of claim 26, wherein the hydrophobic units B_{phobic} comprise units derivable from a hydrophobic monomer comprising:
a vinylaromatic monomer,
a vinyl or vinylidene halide,
a C₁-C₃₀ alkyl ester of an α,β monoethylenically unsaturated acid,
a vinyl or allyl alcohol ester of a carboxylic acid,
an α,β-monoethylenically unsaturated nitrile comprising from 5 to 12 carbon atoms,
an α-olefin,
a conjugated diene, or
a mixture or combination, thereof.

49. The composition of claim 26, wherein the hydrophobic units B_{phobic} comprise units derivable from a hydrophobic monomer comprising:
a styrene
a vinyltoluene,
a vinyl chloride,
a vinylidene chloride,
a methyl, ethyl, butyl, 2-ethylhexyl, isooctyl, lauryl, isodecyl or stearyl acrylate or methacrylate,
a vinyl or allyl acetate, propionate, versatate or stearate,
an acrylonitrile, or
a mixture or combination thereof.

50. The composition of claim 26, wherein the amphiphilic units Bₐₘₚₕᵢ comprise units derivable from an amphiphilic monomer comprising:
an acrylate or methacrylate of a poly(ethoxylated and/or propoxylated) C₃-C₃₀ aliphatic alcohol comprising an aliphatic part that is optionally substituted with one or more hydroxyls,
a poly(ethoxylated and/or propoxylated)polystyrylphenol acrylate or methacrylate,
a poly(ethoxylated and/or propoxylated)alkylphenol acrylate or methacrylate; or
a mixture or combination thereof.

51. The composition of claim 26, wherein:
the surfactant system comprises an anionic surfactant,
the composition comprises at least one salt and/or a structuring agent, and
the composition comprises organized phases of surfactant(s) comprising giant micelles.

52. The composition of claim 26, wherein the surfactant system comprises at least one anionic surfactant and at least one amphoteric surfactant.

53. The composition of claim 26, wherein said composition comprises from 0.01% to 5% by weight of at least one salt.

54. The composition of claim 26, wherein said composition comprises a shampoo, a hair conditioner, a shower gel, a cleansing product for personal hygiene, a face cleanser or a hand cleanser.

55. A process for modify the rheology, texture, suspension properties, and/or foaming properties of a composition comprising the step of adding to the composition at least one copolymer comprising:
units A_{Z} comprising a betaine group comprising a cationic group and an anionic group other than a carboxylate group,
units B comprising hydrophobic units B_{phobic} and/or amphiphilic units Bₐₘₚₕᵢ, and
optionally other hydrophilic units Aₒₜₕₑᵣ.
to an aqueous cosmetic composition comprising a surfactant system and optionally at least one salt.

56. The composition of claim 26, wherein said composition comprises the at least one copolymer in an amount effective to modify the rheology, texture, suspension properties, and/or, foaming properties of the composition.

57. The composition of claim 49, wherein said styrene comprises an α-methylstyrene or para-chioromethylstyrene.
